(19) United States
(12) Patent Application Publication
(10) Pub. No.: US 2025/0057643 A1
(43) Pub. Date: Feb. 20, 2025

(54) MEDICAL IMPLANT AND IMPLANTATION FRAME

(71) Applicants: Smith & Nephew, Inc., Memphis, TN (US); Smith & Nephew Orthopaedics AG, Zug (CH); Smith & Nephew Asia Pacific Pte. Limited

(72) Inventors: Carolyn M. Krasniak, Melrose, MA (US); David A. Yeung, Andover, MA (US); Brendan Jablonski, Boston, MA (US); Rachel S. Keen, Medford, MA (US); Kevin M. Falco, Chelmsford, MA (US)

(73) Assignees: Smith & Nephew, Inc., Memphis, TN (US); Smith & Nephew Orthopaedics AG, Zug (CH); Smith & Nephew Asia Pacific Pte. Limited, (omitted)

(21) Appl. No.: 18/936,354

(22) Filed: Nov. 4, 2024

Related U.S. Application Data
(63) Continuation of application No. PCT/US2023/023826, filed on May 30, 2023.
(60) Provisional application No. 63/347,737, filed on Jun. 1, 2022.

(51) Int. Cl. A61F 2/08; A61F 2/00

(52) U.S. Cl. CPC A61F 2/0811; A61F 2/0095; A61F 2002/0817; A61F 2002/0858; A61F 2220/0016; A61F 2250/0036; A61F 2250/0039; A61F 2250/0071

(57) ABSTRACT

An implant delivery system for delivering an implant to the body. The implant delivery system may comprise a delivery shaft including a proximal portion and a distal portion and a detachable frame coupled to the distal portion of the delivery shaft. The detachable frame may include a body portion and a plurality of attachment arms extending away from the body portion. At least one soft tissue anchor may be releasably secured to a free end of at least one of the plurality of attachment arms and may be configured to releasably secure an implant to the detachable frame.

### CROSS REFERENCE TO RELATED APPLICATIONS

This application is a continuation of International Application No. PCT/US2023/023826, filed May 30, 2023, which claims the benefit of U.S. Patent Application Ser. No. 63/347,737 filed on Jun. 1, 2022, the disclosures of which are incorporated herein by reference.

### TECHNICAL FIELD

The present disclosure pertains generally, but not by way of limitation, to orthopedic implants and methods of treatment. More particularly, the present disclosure relates to systems and methods for delivering a tendon repair implant, such as one that is engineered for arthroscopic placement over or in the area of a full or partial thickness tear of the supraspinatus tendon of the shoulder.

### BACKGROUND

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear or from overuse of the joint. There is an ongoing need to deliver and adequately position medical implants during an arthroscopic procedure in order to treat injuries to the rotator cuff, rotator cuff tendons, or other soft tissue or tendon injuries throughout a body.

### BRIEF SUMMARY

This disclosure provides design, material, manufacturing method, and use alternatives for implants used for soft tissue repairs such as but not limited to rotator cuff repairs.

One example is an implant delivery system. The implant delivery system includes a delivery shaft including a proximal portion and a distal portion, and a frame coupled to the distal portion of the delivery shaft. The frame includes a body portion and a plurality of attachment arms extending away from the body portion. At least one soft tissue anchor is releasably secured to a free end of at least one of the plurality of attachment arms. An implant is releasably secured to the frame via the at least one soft tissue anchor.

Additionally or alternatively, in another example the at least one soft tissue anchor is configured to remain in a body with the implant.

Additionally or alternatively, in another example the frame has a first surface configured to face away from the implant when the implant is secured to the frame and a second surface configured to face towards the implant when the implant is secured to the frame.

Additionally or alternatively, in another example the at least one tissue anchor comprises a proximal head portion, a distal fixation end, and an intermediate connecting portion extending between the proximal head portion and the distal fixation end.

Additionally or alternatively, in another example the proximal head portion is generally disc-shaped.

Additionally or alternatively, in another example the distal fixation end comprises a first barb and a second barb.

Additionally or alternatively, in another example the intermediate connection portion comprises an elongate rod.

Additionally or alternatively, in another example the proximal head portion is a truncated pyramid.

Additionally or alternatively, in another example the distal fixation end has a barbed “U” shape including a first leg and a second leg.

Additionally or alternatively, in another example the intermediate connection portion forms a bridge between the first and second legs.

Additionally or alternatively, in another example the system further comprises a recess formed in the at least one of the plurality of attachment arms adjacent to the free end thereof.

Additionally or alternatively, in another example the recess extends partially through a thickness of the at least one of the plurality of attachment arms to define a reduced thickness region.

Additionally or alternatively, in another example the system further comprises a through hole extending through the reduced thickness region.

Additionally or alternatively, in another example the recess is configured to receive the proximal head portion of the at least one soft tissue anchor therein.

Additionally or alternatively, in another example the intermediate connection portion extends through the through hole.

Additionally or alternatively, in another example under an applied force, the proximal head portion of the at least one soft tissue anchor is configured to push through the reduced thickness region of the at least one of the plurality of attachment arms to release the at least one soft tissue anchor from the frame.

Additionally or alternatively, in another example the reduced thickness region is configured to fracture.

Additionally or alternatively, in another example the reduced thickness region is configured to deform to allow the proximal head portion to pass through the through hole.

Additionally or alternatively, in another example the proximal head portion further comprises one or more radially extending tabs.

Additionally or alternatively, in another example the system further comprises an aperture formed through the at least one of the plurality of attachment arms adjacent the free end thereof.

Additionally or alternatively, in another example the aperture extends through a thickness of the at least one of the plurality of attachment arms.

Additionally or alternatively, in another example the system further comprises a circumferentially extending groove formed in a side wall of aperture, the circumferentially extending groove positioned between a first surface of the at least one of the plurality of attachment arms and a second surface of the at least one of the plurality of attachment arms.

Additionally or alternatively, in another example the system further comprises at least one axially extending slot formed in a side wall of aperture, the at least one axially extending slot extending from the second surface of the at least one of the plurality of attachment arms to the circumferentially extending groove.

Additionally or alternatively, in another example during assembly of the at least one soft tissue anchor with the at least one of the plurality of attachment arms and deployment of the at least one soft tissue anchor from the at least one of the plurality of attachment arms, the one or more radially extending tabs are configured to slide within the at least one axially extending slot.

Additionally or alternatively, in another example the one or more radially extending tabs are rotatably disposed within the circumferentially extending groove during delivery of the implant to a target location.

Additionally or alternatively, in another example the proximal head portion further comprises one or more radially extending detents.

Additionally or alternatively, in another example the detent extends about a circumference of the proximal head portion.

Additionally or alternatively, in another example the system further comprises an aperture formed through the at least one of the plurality of attachment arms adjacent the free end thereof.

Additionally or alternatively, in another example the aperture extends through a thickness of the at least one of the plurality of attachment arms.

Additionally or alternatively, in another example the system further comprises a retainer, the retainer configured to slide over a free end of the at least one of the plurality of attachment arms.

Additionally or alternatively, in another example the retainer comprises a top side, a bottom side, a first lateral side extending between the top side and the bottom side, and a second lateral side opposite the first lateral side and extending between the top side and the bottom side.

Additionally or alternatively, in another example collectively the top side, the bottom side, the first lateral side, and the second lateral side define a channel that extends from a first end to a second end of the retainer, the channel configured to receive the at least one of the plurality of attachment arms.

Additionally or alternatively, in another example the system further comprises a first opening in the top side of the retainer and a second opening defined in the bottom side of the retainer.

Additionally or alternatively, in another example the first opening has a first diameter and the second opening has a second diameter, the second diameter greater than the first diameter.

Additionally or alternatively, in another example the first and second openings are generally aligned to define a retainer aperture extending from the top side to the bottom side.

Additionally or alternatively, in another example the retainer aperture is aligned with the aperture of the at least one of the plurality of attachment arms.

Additionally or alternatively, in another example the one or more radially extending detents of the proximal head portion are received within a mating recess formed in a side wall of the first opening of the retainer.

Additionally or alternatively, in another example the system further comprises an aperture formed through the at least one of the plurality of attachment arms adjacent the free end thereof.

Additionally or alternatively, in another example the aperture extends through a thickness of the at least one of the plurality of attachment arms.

Additionally or alternatively, in another example the system further comprises a retainer, the retainer configured to slide over a free end of the at least one of the plurality of attachment arms.

Additionally or alternatively, in another example the retainer comprises a top side, a bottom side, a first lateral side extending between the top side and the bottom side, and a second lateral side opposite the first lateral side and extending between the top side and the bottom side.

Additionally or alternatively, in another example collectively the top side, the bottom side, the first lateral side, and the second lateral side define a channel that extends from a first end to a second end of the retainer, the channel configured to receive the at least one of the plurality of attachment arms.

Additionally or alternatively, in another example the system further comprises a first opening in the top side of the retainer and a second opening defined in the bottom side of the retainer.

Additionally or alternatively, in another example the first and second openings are generally circular.

Additionally or alternatively, in another example the first and second openings are generally aligned to define a retainer aperture extending from the top side to the bottom side.

Additionally or alternatively, in another example the first opening has a first diameter and the second opening has a second diameter, the second diameter greater than the first diameter.

Additionally or alternatively, in another example the first diameter of the first opening of the retainer is less than a diameter of the proximal head portion of the at least one soft tissue anchor.

Additionally or alternatively, in another example the retainer aperture is aligned with the aperture of the at least one of the plurality of attachment arms.

Additionally or alternatively, in another example the intermediate connection portion is disposed within the retainer aperture and the aperture of the at least one of the plurality of attachment arms and a bottom surface of the proximal head region of the at least one soft tissue anchor engages the top side of the retainer.

Additionally or alternatively, in another example the retainer is configured to fracture to release the at least one soft tissue anchor.

Additionally or alternatively, in another example the at least one of the plurality of attachment arms comprises a first flexible arm and a second arm adjacent the free end thereof.

Additionally or alternatively, in another example the first and second flexible arms increase in width towards the free end to define a distal opening having a first width and an aperture having a second width, the second width greater than the first width.

Additionally or alternatively, in another example the intermediate connection portion is disposed within the aperture.

Additionally or alternatively, in another example the first and second flexible arms are configured to flex away from a longitudinal axis of the at least one of the plurality of attachment arms to receive and deploy the at least one soft tissue anchor in response to an applied force.

Additionally or alternatively, in another example in the absence of an applied force, the first and second arms are configured to return to an unbiased configuration.

Additionally or alternatively, in another example the first and second arms are configured to engage a recess formed in a side wall of the proximal head portion.

Additionally or alternatively, in another example the proximal head portion comprises a top side, a bottom side, a first lateral side extending between the top side and the bottom side, and a second lateral side opposite the first lateral side and extending between the top side and the bottom side.

Additionally or alternatively, in another example collectively the top side, the bottom side, the first lateral side, and the second lateral side define a channel that extends from a first end to a second end of the proximal head portion, the channel configured to receive the at least one of the plurality of attachment arms.

Additionally or alternatively, in another example the distal fixation end comprises a first barb and a second barb.

Additionally or alternatively, in another example the intermediate connection portion comprises an elongate rod.

Additionally or alternatively, in another example the wherein the at least one of the plurality of attachment arms comprises a first flexible arm and a second flexible arm adjacent the free end thereof.

Additionally or alternatively, in another example the first and second flexible arms define a gap therebetween.

Additionally or alternatively, in another example the first and second flexible arms each include a proximal portion which extends generally parallel to a longitudinal axis the at least one of the plurality of attachment arms and a distal portion terminating in a free end which extends at a non-parallel angle to the longitudinal axis of the at least one of the plurality of attachment arms.

Additionally or alternatively, in another example the free ends of the first and second flexible arms may each curve away from a longitudinal axis of the at least one of the plurality of attachment arms such that a first width of the at least one of the plurality of attachment arms adjacent the free ends is greater than a second width of the at least one of the plurality of attachment arms adjacent to the proximal portion of flexible arms.

Additionally or alternatively, in another example the first and second flexible arms are configured to flex inwards towards a longitudinal axis of the at least one of the plurality of attachment arms under an applied force.

Additionally or alternatively, in another example the first and second flexible arms are configured to flex inwards to dispose the first and second flexible within the channel of the proximal head portion of the at least one soft tissue anchor and to deploy the at least one soft tissue anchor from the at least one of the plurality of attachment arms.

Additionally or alternatively, in another example the at least one of the plurality of attachment arms comprises a raised detent extending from a surface thereof adjacent the free end.

Additionally or alternatively, in another example the raised detent comprises a distal side and a proximal side.

Additionally or alternatively, in another example the distal side of the raised detent increases in height in a proximal direction.

Additionally or alternatively, in another example the proximal side of the raised detent extends at an angle orthogonal to the surface of the at least one of the plurality of attachment arms.

Additionally or alternatively, in another example the at least one of the plurality of attachment arms is disposed within the channel of the proximal head portion of the at least one soft tissue anchor.

Additionally or alternatively, in another example the raised detent is positioned outside of the channel adjacent to the first end of the proximal head portion.

Additionally or alternatively, in another example the raised detent is configured to deform when a lateral pulling force exerted on the detachable frame allowing the at least one of the plurality of attachment arms to pass through the channel of the proximal head portion of the at least one soft tissue anchor.

Additionally or alternatively, in another example the proximal head portion further comprises a first detent and a second detent extending into the channel from the top side or the bottom side of the proximal head portion.

Additionally or alternatively, in another example the at least one of the plurality of attachment arms comprises a first recess and a second recess formed in a surface thereof, the second recess laterally spaced from the first recess.

Additionally or alternatively, in another example the first and second detents of the proximal head portion are configured to mate with the first and second recesses of the at least one of the plurality of attachment arms when the at least one of the plurality of attachment arms is disposed within the channel of the proximal head portion.

Additionally or alternatively, in another example the first and second detents are configured to disengage from the first and second recesses when a lateral pulling force exerted on the detachable frame allowing the at least one of the plurality of attachment arms to pass through the channel of the proximal head portion of the at least one soft tissue anchor.

Additionally or alternatively, in another example the system further comprises an aperture formed through the at least one of the plurality of attachment arms adjacent the free end thereof.

Additionally or alternatively, in another example the aperture extends through a thickness of the at least one of the plurality of attachment arms.

Additionally or alternatively, in another example the aperture is generally rectangular.

Additionally or alternatively, in another example the system further comprises a retainer, the retainer configured to slide over a free end of the at least one of the plurality of attachment arms.

Additionally or alternatively, in another example the retainer comprises a top side, a bottom side, a first lateral side extending between the top side and the bottom side, and a second lateral side opposite the first lateral side and extending between the top side and the bottom side.

Additionally or alternatively, in another example collectively the top side, the bottom side, the first lateral side, and the second lateral side define a channel that extends from a first end to a second end of the retainer, the channel configured to receive the at least one of the plurality of attachment arms.

Additionally or alternatively, in another example the system further comprises a first opening in the top side of the retainer and a second opening defined in the bottom side of the retainer.

Additionally or alternatively, in another example the first and second openings are generally aligned to define a retainer aperture extending from the top side to the bottom side.

Additionally or alternatively, in another example the first opening has a first width and length and the second opening has a second width and length, the second width and length greater than the first width and length.

Additionally or alternatively, in another example the first width and length of the first opening of the retainer is less than a width and a length of an upper face of the proximal head portion of the at least one soft tissue anchor.

Additionally or alternatively, in another example the retainer aperture is aligned with the aperture of the at least one of the plurality of attachment arms.

Additionally or alternatively, in another example the intermediate connection portion is disposed within the retainer aperture and the aperture of the at least one of the plurality of attachment arms and a plurality of side faces of the proximal head region of the at least one soft tissue anchor engages the top side of the retainer.

Additionally or alternatively, in another example the retainer is configured to fracture to release the at least one soft tissue anchor.

Additionally or alternatively, in another example the retainer is configured to flex to release the at least one soft tissue anchor.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates a cross-section of an anterior view of a shoulder of a patient;

FIG. 2 illustrates a shoulder including a head of the humerus mating with the glenoid fossa of the scapula at a glenohumeral joint and an implant affixed to a tendon;

FIG. 3A illustrates an example implant delivery device attached to an implant;

FIG. 3B illustrates an example delivery device attached to an implant;

FIG. 3C illustrates an example delivery device attached to an implant;

FIG. 4 illustrates an example implant delivery device attached to an implant;

FIG. 5 illustrates an example frame attached to an example implant;

FIG. 6A illustrates a detailed view of an illustrative soft tissue anchor assembled with a frame and implant;

FIG. 6B illustrates a cross-sectional view of the illustrative soft tissue anchor and attachment arm of FIG. 6A;

FIG. 7A illustrates an exploded perspective view of another illustrative attachment arm of a frame and soft tissue anchor;

FIG. 7B illustrates a cross-sectional view of the illustrative attachment arm of FIG. 7A;

FIG. 7C illustrates a top view of the soft tissue anchor assembled with the illustrative attachment arm of FIG. 7A;

FIG. 8A illustrates a perspective view of another illustrative attachment arm of a frame and a soft tissue anchor;

FIG. 8B illustrates an exploded perspective view of the illustrative attachment arm of a frame and soft tissue anchor of FIG. 8A;

FIG. 8C illustrates a cross-sectional view of the illustrative attachment arm of FIG. 8A.

FIG. 8D illustrates a cross-sectional view of an alternative retainer assembled with the attachment arm;

FIG. 8E illustrates a perspective view of the illustrative retainer of FIG. 8D after a soft tissue anchor has been deployed therefrom;

FIG. 9A illustrates an exploded perspective view of another illustrative attachment arm of a frame and soft tissue anchor;

FIG. 9B illustrates a perspective view of the illustrative attachment arm of FIG. 9A;

FIG. 9C illustrates a cross-sectional view of an alternative soft tissue anchor for use with the attachment arm of FIGS. 9A and 9B;

FIG. 10A illustrates an exploded perspective view of another illustrative attachment arm of a frame and soft tissue anchor;

FIG. 10B illustrates a perspective view of the illustrative attachment arm of a frame assembled with the soft tissue anchor of FIG. 10A;

FIG. 11A illustrates an exploded perspective view of another illustrative attachment arm of a frame and soft tissue anchor;

FIG. 11B illustrates a side view of the illustrative attachment arm of a frame assembled with the soft tissue anchor of FIG. 11A and an implant;

FIG. 12A illustrates an exploded perspective view of another illustrative attachment arm of a frame and soft tissue anchor;

FIG. 12B illustrates a partial cross-sectional view of the illustrative attachment arm of a frame assembled with the soft tissue anchor of FIG. 12A;

FIG. 13A illustrates a perspective view of another illustrative attachment arm of a frame and soft tissue anchor;

FIG. 13B illustrates an exploded perspective view of the illustrative attachment arm of a frame, retainer and soft tissue anchor of FIG. 13A; and

FIG. 13C is a cross-sectional view of the illustrative attachment arm of FIG. 13A.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear, or from overuse of the joint. Current repair procedures may attempt to alleviate impingement or make room for movement of the tendon to prevent further damage and relieve discomfort but do not repair or strengthen the tendon. An accepted treatment for rotator cuff tears may include reattaching the torn tendon to the humeral head using sutures. Additionally, in treating rotator cuff tears, an accepted practice may also include the placement of a scaffold over the repaired tendon to mechanically reinforce the repaired tendon. Therefore, there is an ongoing need to deliver and adequately position medical implants during an arthroscopic procedure in order to treat injuries to the rotator cuff, rotator cuff tendons, or other soft tissue or tendon injuries throughout a body.

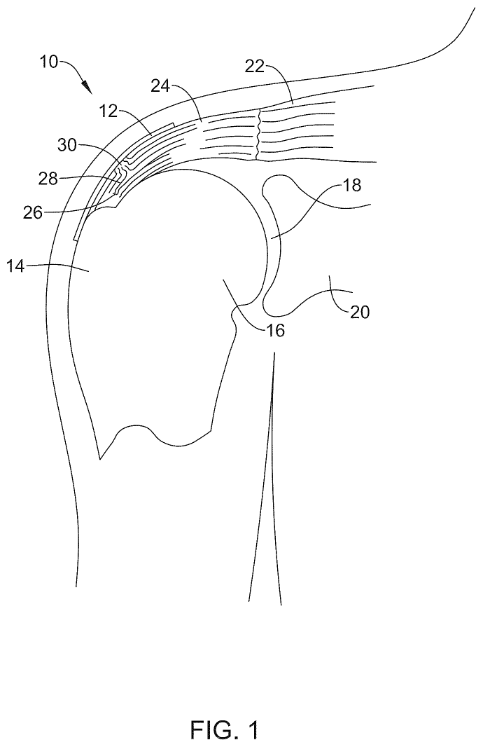

FIG. 1 shows a cross-sectional view of a shoulder 10 including an example implant 12. Shoulder 10 further shows a head 14 of a humerus 16 mating with a glenoid fossa 18 of scapula 20. The glenoid fossa 18 comprises a shallow depression in scapula 20. A supraspinatus tendon 22 is also shown. These muscles (along with others) control the movement of the humerus 16 relative to the scapula 20. A distal tendon 24 of the supraspinatus tendon 22 meets the humerus 16 at an insertion point 26.

In FIG. 1, the distal tendon 24 includes a damaged portion 28 located near the insertion point 26. The damaged portion 28 includes a tear 30 extending partially through the distal tendon 24. The tear 30 may be referred to as a partial thickness tear. The depicted partial thickness tear 30 is on the bursal side of the tendon, however, the tear may also be on the opposite or articular side of the distal tendon 24 and/or may include internal tears to the distal tendon 24 not visible on either surface. In other instances, the tear 30 may be a full thickness tear.

FIG. 1 further illustrates that the tendon repair implant 12 has been placed over the partial thickness tear 30. In this example, the tendon repair implant 12 is placed on the bursal side of the tendon regardless of whether the tear is on the bursal side, articular side or within the tendon. Further, the tendon repair implant 12 may overlay multiple tears.

Although one possible treatment site is described in the figures as being located in a shoulder joint, the implant 12 may be used at a variety of different treatment sites, such as the hip, knee, ankle, etc. Furthermore, the implant 12 may be used for any of a variety of soft tissue repairs, such as but not limited to the gluteus medius, which is a large fan-shaped muscle located in the posterior hip, and the hip capsule, which is also in the hip. The implant 12 may be used in treating soft tissue injuries in the knee, such as but not limited to ligaments such as the ACL (anterior cruciate ligament), MCL (medial collateral ligament) and the PCL (posterior cruciate ligament) and tendons such as the hamstring tendons, the quadriceps tendon and the patellar tendon. The implant 12 may be used in treating soft tissue injuries in the Achilles tendon. The implant 12 may be used in treating soft tissue injuries for any areas of the body that will accommodate the implant 12.

In some instances, delivery of an implant 12 (e.g., a sheet-like implant) to a target site of a patient may require a physician to create an incision in the patient sufficient to access the target implant site. After creating this “access site,” the physician may insert an implant delivery system through the access site and position the distal end of the implant delivery system adjacent the target implant site. The physician may then manipulate the implant delivery system to deploy an implant out of a delivery sheath adjacent the target implant site.

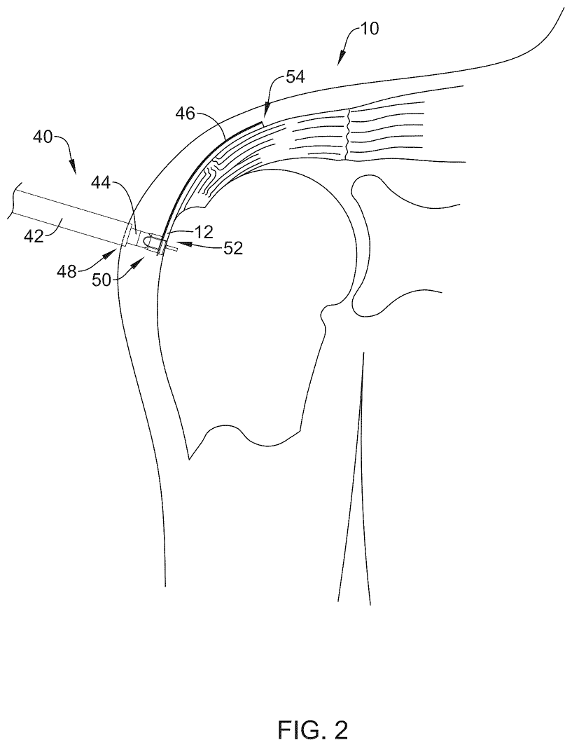

For example, FIG. 2 provides a perspective view of an implant delivery system 40 extending through the shoulder 10 of a patient. FIG. 2 shows the implant delivery system 40 deployed adjacent a target site (e.g., a tear in the supraspinatus tendon). In at least some embodiments, the implant delivery system 40 includes a sheath member 42 (e.g., a cannula) including a proximal portion (not shown), a distal portion 48, and a lumen extending within at least a portion of the cannula 42. Further, the implant delivery system 40 may include a delivery shaft 44 extending within the lumen of the sheath member 42 and longitudinally movable relative thereto.

The delivery shaft 44 may include a proximal portion (not shown) extending out of the proximal portion of the sheath member 42 and/or otherwise manipulatable relative to the sheath member 42 by a user. Additionally, in some examples, the proximal portion of the delivery shaft 44 and/or the sheath member 44 may be coupled to a handle member (not shown). The handle member may be utilized to manipulate the delivery shaft 44. For example, the handle member may be utilized to impart a rotational force to the delivery shaft 44.

In addition, the delivery shaft 44 may include a distal portion 50 extending out of the distal portion 48 of the sheath member 42. Further, the delivery shaft 44 may include a lumen extending therein. The lumen of the delivery shaft 44 may extend along a portion or the entire length of the delivery shaft 44 (e.g., from the distal portion 50 to the proximal portion of the delivery shaft 44).

The delivery system 40 may further include a detachable frame member 46 attached to the distal portion 50 of the delivery shaft 44, which in some instances may be detachable from the delivery shaft 44. As shown in FIG. 2, the detachable frame 46 may be attached to an implant 12 (e.g., a sheet-like implant) for delivery to the implantation site, and subsequent placement thereon. For purposes of the discussion herein, the combined structure including the frame 46 and the implant 12 may be defined as having a proximal end 52 and a distal end 54 as illustrated in FIG. 2.

When initially positioning the frame 46 and implant 12 adjacent a target site, a clinician may orient the frame 46 and the implant 12 (for example, via a handle member attached to a proximal portion of the delivery shaft 44) such that the proximal portion 52 (i.e., the lateral portion) may be adjacent (e.g., overlaid) on a portion of the humerus (e.g., on the bone), while the distal portion 54 (i.e., the medial portion) of the frame 46 and the implant 12 may overlay the distal tendon 24.

As described above, delivery of implant delivery system 40 may include the insertion of delivery sheath 42 through an access site (e.g., incision) and advancement to a target site. After positioning the distal end 48 of the delivery sheath 42 proximate the target site, a clinician may deploy the detachable frame 46 in combination with the implant 12 out of the lumen located within and along the distal portion 48 of the delivery sheath 42, such as by retracting the delivery sheath 42 relative to the delivery shaft 44 and the frame 46, and positioning the implant 12 and the frame 46 over the target site.

Prior to deployment, the detachable frame 46 and the implant 12 combination may be contained (e.g., housed) within the lumen of the delivery sheath 42 for subsequent deployment distally out distal opening of the delivery sheath 42. The combination of the detachable frame 46 and the implant 12 may wrap and/or fold upon itself such that it may be positioned within the lumen of the delivery sheath 42. Alternatively, the detachable frame 46 and the implant 12 may wrap and/or fold around the implant delivery shaft 44 while disposed within the delivery sheath 42.

In some cases, the detachable frame 46 may be formed as a monolithic structure by being formed (e.g., machined, cut, shaped, stamped, laser-cut, etc.) as a unitary structure from a single piece of material. It is contemplated that the detachable frame 46 may be constructed using alternative materials and/or manufacturing methodologies. For example, the frame 46, or portions thereof, may be constructed from a polymeric material, a ceramic material, and/or other various materials. Additionally, the frame 46 may be manufactured via an injection molding or alternative polymer manufacturing methodologies. Alternatively, the frame 46 may be formed through a 3-D printing process, if desired. Further, different portions of the frame 46 may be made from a variety of materials and combined using alternative methodologies. Variations of combining different materials with different portions of the frame 46 are contemplated.

For simplicity purposes, when combined with an example implant 12, the frame 46 may be defined as having a first surface that faces away from the implant 12 when the implant 12 is attached to the frame 46 (e.g., a first surface that faces away from a target site in the body) and a second surface that faces the example implant 12 (e.g., a second surface that faces a target site in the body). In some instances, attachment apertures 70 may extend from the first surface to the second surface. In other words, in some instances, attachment apertures 70 may be defined as holes and/or openings that extend through the thickness of the frame 46 from the first surface of the frame 46 that faces away from the implant 12 to the second surface of the frame 46 that faces toward the implant 12.

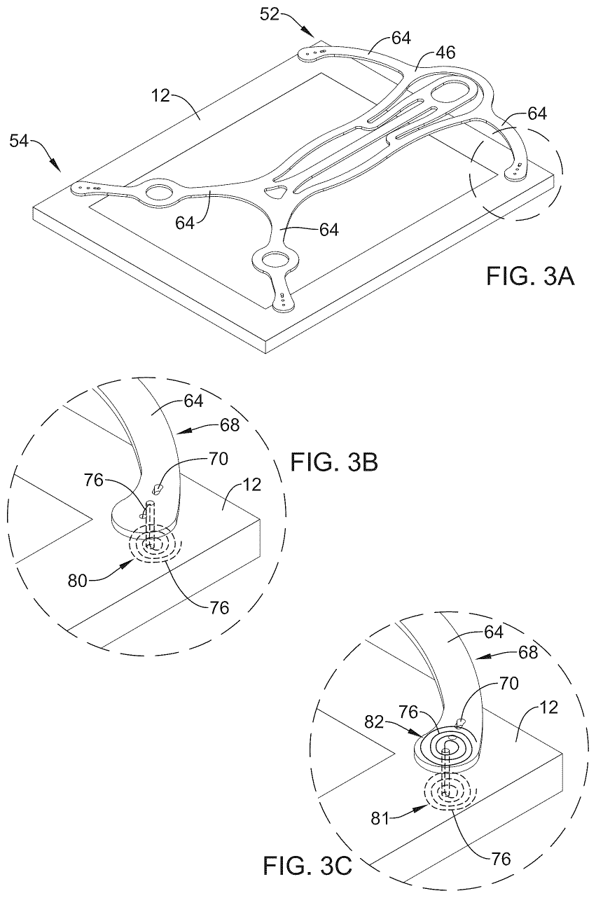

Attachment apertures 70 may be utilized to attach and/or couple frame 46 to an example implant 12. FIG. 3A shows an example frame 46 attached to an example implant 12. Further, FIG. 3 shows example frame 46 attached to example implant 12 at the distal or free end of each of four attachment arms 64, respectively. Attachment of free distal ends of attachment arms 64 to implant 12 may be made by any desired attachment mechanism.

FIG. 3B shows a detailed view of a portion of the proximal portion 54 of a frame 46 attached to an implant 12. Further, FIG. 3B shows example attachment arm 64 including a distal portion 68. Three attachment apertures 70 are positioned along the distal portion 68 of the attachment arm 64. Additionally, FIG. 3B shows an example attachment member (e.g. wire) 76 extending between and through one or more of the attachment apertures 70 located on the distal portion 68 of the attachment arms 64.

The attachment members 76 may be one of several structures and/or techniques contemplated to attach the example frame 46 to the example implant 12. As shown in FIG. 3B, the attachment member 76 may be positioned, looped, wound and/or threaded through one or more attachment apertures 70 such that the attachment member 76 is prevented from being pulled away from the distal portion 68 of the attachment arm 64. In other words, winding the attachment member 76 through one or more of the attachment apertures 70 may effectively affix the attachment member 76 onto the attachment arm 64. In other words, it is contemplated that the attachment member 76 may be affixed to the distal portion 68 of the attachment arms 64 (via attachment apertures 70, for example) without having either end of the attachment member 76 directly attached (e.g., welded, tied, etc.) to any structure (e.g., frame 46). In some instances, the attachment member 76 may be wrapped and/or looped through the attachment apertures 70 one or more times to provide a friction fit and/or resistive tension to unraveling or unwinding as a withdrawal force is applied to the attachment member 76. In other instances, the implant may be attached to the attachment arms 64 in another fashion, such as with other attachment members attached to and/or extending from the attachment arms 64.

While FIG. 3B shows a single attachment member 76 extending between two attachment apertures 70, it is contemplated that the attachment member 76 may extend and/or wrap between two or more of the attachment apertures 70. For example, it is contemplated that the attachment member 76 may be woven (e.g., over-and-under) through three apertures 70 in order to lock the attachment member 76 to the distal end 68 of the attachment arm 64.

The above discussion and the forgoing examples are not intended to limit the disclosure to using an attachment member (e.g., wire, thread, cable, etc.) to attach the frame 46 to the implant 12. Rather, a variety of methodologies may be utilized to attach the frame 46 to the implant 12. For example, adhesives may be used alone or in combination with another attachment mechanism to attach the frame 46 to the implant 12. Additionally, a variety of injection molding techniques may be employed to attach the frame 46 to the implant 12. Further, combinations of the disclosed techniques may be used to attach the frame 46 to the implant 12. For example, an attachment member 76 may be used in conjunction with an adhesive to attach the frame 46 to the implant 12 without having to wind the attachment member 76 through the attachment apertures 70.

As stated above, it is contemplated in the examples discussed herein that the frame 46 may be able to be “detached” from the implant 12. For example, the frame 46 may be configured to detach from the implant 12 after the implant 12 has been affixed to a target site in the body. Therefore, it can be appreciated that in some examples disclosed herein, the frame member 46 may be temporarily attached to the implant 12. For example, the frame member 46 may be coupled, affixed or attached to the implant 12 while positioned within the delivery sheath 42, deployed out of the delivery sheath 42 and maneuvered into position relative to a target site. Once positioned at the target site (e.g., along the tendon and/or humeral head), the implant 12 may be affixed to the target site via bio-adhesives, tissue anchors, and/or sutures. However, once the implant 12 has been affixed to the target site, the frame 46 may be pulled away (e.g., detached) from the implant 12 and removed from the body. As will be described in more detail herein, in some cases, the implant 12 may be releasably coupled to the frame 46 via one or more tissue anchors, which are also utilized to secure the implant 12 to tissue at the target site, and thereafter released from the frame 46 to remain with the implant at the target site at the conclusion of the medical procedure.

FIG. 3B shows an example attachment configuration which may allow the frame 46 to detach from the implant 12. FIG. 3B shows attachment member 76 wound in a spiral pattern 80 along the surface of implant 12 facing a target site. In other words, attachment member 76 may form a spiral pattern 80 that remains in a plane substantially parallel to the plane of the surface of implant 12 which faces a target site. Further, it can be appreciated that attachment member 76 may extend from the side of attachment arm 64 facing away from implant 12, through the combined thickness of the attachment arm 64 and implant 12, eventually exiting implant 12 on the surface of implant 12 facing a target site. Further, it can be appreciated that the spiral pattern 80 shown in FIG. 3B is one of a variety of configurations for which attachment member 76 may be wound in order to prevent frame 46 from prematurely releasing from implant 12.

Attachment member 76 may have a first end secured to a free distal end of attachment arm 64 positioned on a first side of implant 12 and have a second end positioned on a second, opposite side of implant 12. In some instances, attachment member 76 may extend through implant 12 from the first side of implant 12 to the second side of implant 12. However, in other instances, attachment member 76 may extend around an edge of implant 12 from the first side of implant 12 to the second side of implant 12.

In some cases, the attachment member 76 may be configured to be detached from implant 12 upon application of a threshold level of force. For example, the spiral pattern 80 shown in FIG. 3B may provide the frame 46 the ability to detach from the implant 12 when a threshold “pull-away force” is applied to the frame 46. For example, after the implant 12 is affixed to a target site, a clinician may apply a force to the frame 46 (via a tether, for example) such that the frame 46 is pulled away from the implant 12. Provided the force is great enough (e.g., the threshold force is met), the attachment members 76 (e.g., spiral portion 80 of the attachment member 76 shown in FIG. 3B) may be unwound and pulled back through the “body” (e.g., thickness) of the implant 12, thereby releasing the frame 46 from the implant 12. In other words, provided a threshold pull-away force is applied to the frame 46, the attachment member 76 forming the spiral 80 shown in FIG. 3B may unwind and pull back through the implant 12.

FIG. 3C shows another example method to attach the frame 46 to an example implant 12. As shown in FIG. 3C, the attachment member 76 may include a spiral 81 positioned on the surface of the implant 12 which faces away from a target site (similar to the spiral 80 shown in FIG. 3B). Additionally, FIG. 3C shows that the attachment member 76 may include a second spiral 82 positioned on the surface of an attachment arm 68 that faces away from the implant 12. In other words, FIG. 3C shows two spirals 81/82 formed at opposite ends of the attachment member 76 and positioned on both the attachment arm 64 facing away from the implant 12 (e.g., spiral 82 of FIG. 3C) and on the side of the implant 12 lying along a treatment site (e.g., spiral 81 of FIG. 3C). The configuration of spirals 81/82 may provide a frame 46 with a “releasable” connection to the implant 12 similar to that discussed with respect to FIG. 3B.

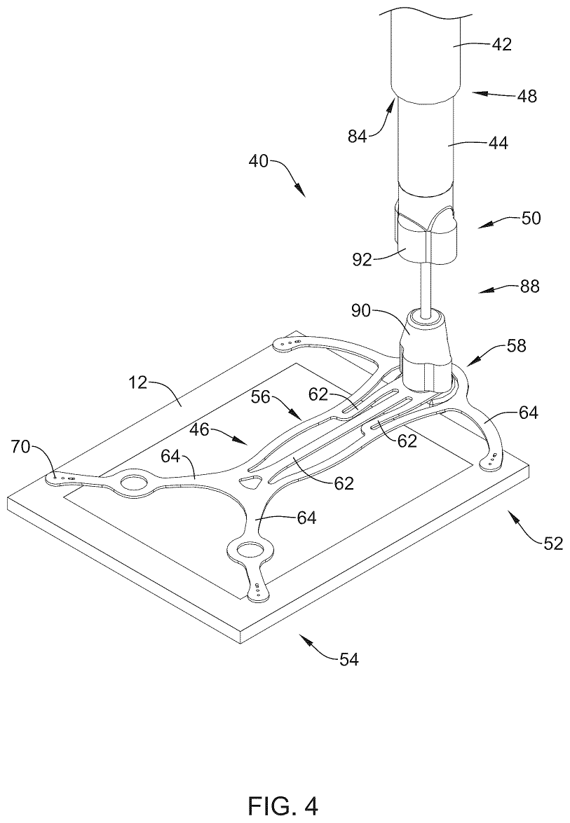

FIG. 4 shows an example frame 46 coupled to an example implant 12 via the attachment members 76 as described above. Further, FIG. 4 shows the frame 46 in combination with the implant 12 coupled to an example implant delivery system 40. Similar to that discussed with respect to FIG. 2, the implant delivery system 40 includes implant delivery shaft 44 extending through an example lumen 84 of an example delivery sheath 42.

Further, FIG. 4 shows the delivery shaft 44 coupled to the frame 46 via a connection assembly 88. The connection assembly 88 may include a first connection member 90 attached to the head portion 58 of the frame 46 and a second connection member 92 attached to the distal end 50 of the delivery shaft 44. While FIG. 4 does not directly show the first connection member 90 attached directly to the second connection member 92, it can be appreciated that the first and second connection members 90/92 of connection assembly 88 may form a mating connection. For example, in some instances, the first connection member 90 may form a male connection member while the second connection member 92 may form a mating female connection member. In other words, in some examples the second connection member 92 may include a cavity which is configured to extend over and allow the first connection member 90 to be inserted therein. In other instances, the first connection member 90 may be a female connection member, while the second connection member 92 may be a mating male connection member.

Additionally, as shown in FIG. 4, it is contemplated that second connection member 92 may disengage or decouple from the first connection member 90. For example, in some instances the connection assembly 88 (including first and second connection members 90/92) may be defined as a “quick release” connection assembly, or otherwise decoupling connection assembly. It is further contemplated that a variety of design configurations may be employed to engage/disengage (i.e., couple/decouple) the first and second connection members 90/92 from one another. For example, the first and second connection members 90/92 may be coupled via a threaded connection, friction fit, spring loaded connection, bayonet connection, movable collar or other actuation mechanism, or the like. Further, the connection member 90/92 may be engaged/disengaged by an operator of the device.

In some instances, the delivery shaft 44 may be attached (via the connection assembly 88, for example) to the head portion 58 of the frame member 46. As shown in FIG. 4, the first connection member 90 of the connection assembly 88 may attach to head portion 58 via an aperture 60 (not shown). In some instances, the first connection member 90 may be attached to the head portion 58 of the frame member 46 via a variety of mechanical fastening means (e.g., injection molding, encapsulation, bonding, etc.)

As discussed above, in some instances, a physician may insert the implant delivery system 40 (including the delivery sheath 42, the delivery shaft 44, the frame 46 and the implant 12) through an incision and position the distal end of the implant delivery system 40 adjacent a target implant site (e.g., torn tendon). Once adjacent the target site, the physician may manipulate the implant delivery shaft 44 to advance the implant 12 (while attached to the detachable frame 46) out of the delivery sheath 42 adjacent the target implant site. For example, the physician may retract the delivery sheath 42 proximally relative to the delivery shaft 44 and the frame 46 and/or may advance the delivery shaft 44 and the frame 46 distally relative to the delivery sheath 42.

FIG. 4 shows the frame 46 and the implant 12 deployed from the distal portion 48 of the delivery sheath 42. In some instances, the frame 46 and the implant 12 may have a substantially concave shape with respect to the delivery sheath 42. It can be appreciated that the concave shape of the frame member 46 and the implant 12 may facilitate positioning the implant 12 along the generally rounded shape of the humeral head of the human shoulder.

However, when positioned in the delivery sheath 42 (e.g., prior to deployment) the frame 46 and the implant 12 may be wrapped around the delivery shaft 44 in a convex configuration. Therefore, the frame 46 and the implant 12 may shift from a first convex configuration (while wrapped tightly around the delivery shaft 44 within the lumen 84 of the delivery sheath 42) to a second concave configuration when advanced (e.g., deployed) out of the sheath 42.

In other words, the frame 46 and the implant 12 may be attached to the delivery shaft 44 via the connection assembly 88 when positioned within the lumen 84 of the delivery sheath 42. In one example, when positioned within the delivery sheath 42, the frame 46 and implant 12 may wrap, or extend around, the delivery shaft 44. The position of the frame 46 and implant 12 may be in a convex configuration with respect to the distal end 50 of the delivery shaft 44. As the frame 46 and implant 12 are deployed out of the distal end 50 of the delivery shaft 44, the frame 46 and implant 12 may “shift” from a convex configuration to a concave configuration (as viewed with respect to the distal end 50 of delivery shaft 44). Additional details regarding the frame 46 and the implant delivery system 40 may be found in U.S. Pat. No. 10,314,689, which is incorporated by reference herein in its entirety.

The implant 12 and frame 46 described above may require a delivery system 40 to position the implant 12 in the desired location followed by separate delivery systems to deploy the tendon anchors and the bone anchors to secure the implant 12 to underlying tendon tissue and bone, respectively. In some cases, soft tissue anchors may be delivered one at a time using an anchor inserter which requires the user to re-load each individual soft tissue anchor outside of the arthroscopic space. As such, the anchor inserter may be inserted into and removed from the patient's shoulder (or other implant location) up to eight times or more or as many times as there are soft tissue anchors. Bone anchors may be deployed in a similar manner to soft tissue anchors with an individual single-use punch required per bone anchor deployment. It is contemplated that the soft tissue anchors may be pre-assembled with the implant and/or the frame 46 to reduce the number of procedural steps and/or to reduce to the portal/incision size requirement.

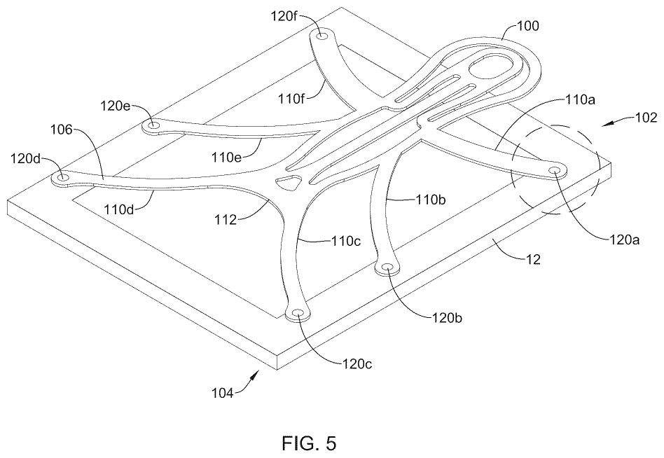

FIG. 5 illustrates another example frame member 100 configured to be releasably attached to the implant 12. The frame 100 may be configured to be delivered to a target location within a body in a manner similar to that described above with respect to frame 46. For purposes of the discussion herein, the combined structure including the frame 100 and the implant 12 may be defined as having a proximal end 102 (i.e., lateral end) and a distal end 104 (i.e., medial end). When initially positioning the frame 100 and implant 12 adjacent a target site, a clinician may orient the frame 100 and the implant 12 (for example, via a handle member attached to a proximal portion of the delivery shaft) such that the proximal portion 102 (i.e., lateral end) may be adjacent (e.g., overlaid) on a portion of the humerus (e.g., on the bone), while the distal portion 104 (i.e., medial end) of the frame 100 and the implant 12 may overlay the tendon.

Prior to deployment, the detachable frame 100 and the implant 12 combination may be contained (e.g., housed) within the lumen of the delivery sheath for subsequent deployment distally out the distal opening of the delivery sheath. The combination of the detachable frame 100 and the implant 12 may wrap and/or fold upon itself such that it may be positioned within the lumen of the delivery sheath. Alternatively, the detachable frame 100 and the implant 12 may wrap and/or fold around the implant delivery shaft while disposed within the delivery sheath.

In some cases, the detachable frame 100 may be formed as a monolithic structure by being formed (e.g., machined, cut, shaped, stamped, laser-cut, etc.) as a unitary structure from a single piece of material. It is contemplated that the detachable frame 100 may be constructed using alternative materials and/or manufacturing methodologies. For example, the frame 100, or portions thereof, may be constructed from a polymeric material, a ceramic material, and/or other various materials. Additionally, the frame 100 may be manufactured via an injection molding or alternative polymer manufacturing methodologies. Alternatively, the frame 100 may be formed through a 3-D printing process, if desired. Further, different portions of the frame 100 may be made from a variety of materials and combined using alternative methodologies. Variations of combining different materials with different portions of the frame 100 are contemplated.

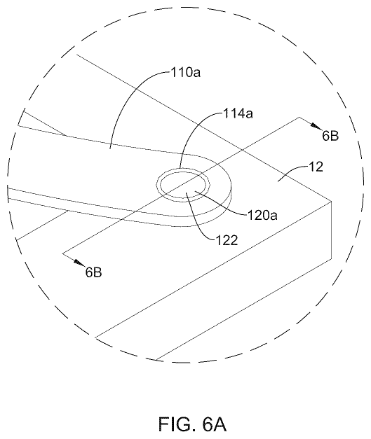

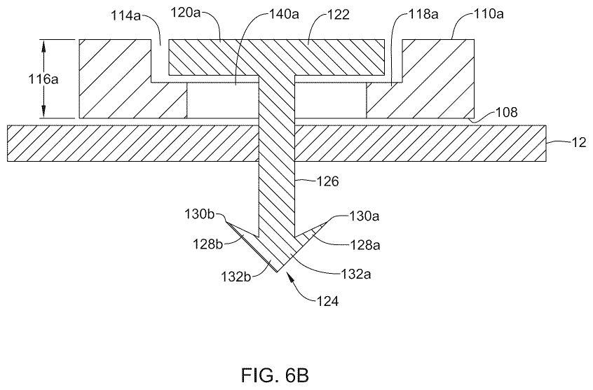

For simplicity purposes, when combined with an example implant 12, the frame 100 may be defined as having a first surface 106 that faces away from the implant 12 when the implant 12 is attached to the frame 100 (e.g., a first surface that faces away from a target site in the body) and a second surface 108 (see, for example, FIG. 6B) that faces or is juxtaposed with the example implant 12 (e.g., a second surface that faces a target site in the body). The frame 100 may include a plurality of attachment arms 110a, 110b, 110c, 110d, 110e, 110f (collectively, 110) extending away from a body portion 112. While the frame 100 is illustrated as including six attachment arms 110, it is contemplated that the frame 100 may include fewer than six (e.g., one, two, three, four, or five) or more than six (e.g., seven, eight, or more) attachment arms 110, as desired. In some cases, the number of attachment arms 110 may be correlated to a number of tissue anchors used to secure the implant 12, as will be described in more detail herein. Soft tissue anchors 120a, 120b, 120c, 120d, 120e, 120f (collectively, 120) may be positioned adjacent a distal or free end of one or more of the attachment arms 110. In some embodiments, each attachment arm 110 may include a soft tissue anchor 120. However, this is not required. In some cases, not all of the attachment arms 110 include a soft tissue anchor 120. For example, some attachment arms 110 may be free from a tissue anchor or other attachment mechanism, or may include an attachment mechanism configured to remain with the frame 100, such as, but not limited to those described in U.S. Pat. No. 10,314,689, which is incorporated by reference herein in its entirety. In some instances, the attachment arms 110b, 110c, 110d and 110e, which overlay soft tissue (e.g., tendon), each include a soft tissue anchor 120, while the attachment arms 110a and 110f, which overlay bone (e.g., the humeral head) each utilize an attachment mechanism configured to remain with the frame 100, as described above.

The soft tissue anchor 120 may be utilized to attach and/or couple the frame 100 to an example implant 12 during delivery of the implant 12 to a treatment site. In some cases, the soft tissue anchors 120 may also anchor the implant 12 to a bodily tissue once positioned at the treatment site. For example, the soft tissue anchor 120 or portions thereof may secure the implant 12 to soft tissue (e.g., tendon) and remain in the body with the implant 12 after the frame 100 has been removed from the treatment site. FIG. 5 shows an example frame 100 attached to an example implant 12. Further, FIG. 5 shows the example frame 100 attached to example implant 12 at the distal or free end of each of six attachment arms 110, respectively. Attachment of free distal ends of attachment arms 110 to implant 12 may be made by any desired attachment mechanism.

FIG. 6A shows a detailed view of an illustrative soft tissue anchor 120a assembled with the frame 100 and implant 12. FIG. 6B is a cross-sectional view of the illustrative soft tissue anchor 120a and attachment arm 110a, taken at line 6B-6B of FIG. 6A. While the soft tissue anchor 120a is described with respect to a particular attachment arm 110a, it should be understood the soft tissue anchor 120a may be provided in any of the attachment arms 110, as desired. The soft tissue anchor 120a may include a proximal head portion 122 configured to hold down the implant 12 and a distal fixation end 124 configured to penetrate the tissue. An elongate intermediate connecting portion or shank 126 may extend between the proximal head portion 122 and the distal fixation end 124. It is contemplated that the proximal head portion 122 may be a generally planar component having a width greater than a width of the intermediate connecting portion or shank 126. In some cases, the proximal head portion 122 may be generally disc-shaped having a generally circular cross-sectional shape. However, this is not required. The proximal head portion 122 may have other cross-sectional shapes, such as, but not limited, square, rectangular, elliptical, polygonal, etc.

The distal fixation end 124 may be generally “V” shaped or pointed, and may include a first barb 128a and a second barb 128b (collectively, 128). The distal fixation end 124 may be shaped such that proximal retraction of the soft tissue anchor 120a is prevented or hindered once the distal fixation end 124 is assembled with the frame 100 and/or body tissue. For example, the barbs 128 may each include a proximal end 130a, 130b (collectively, 130) radially spaced from the intermediate connecting portion or shank 126 and a distal end 132a, 132b in contact with or coupled to the distal fixation end 124. In some cases, fewer than two or more than two barbs 128 may be provided. In some cases, the proximal ends 130 of the barbs 128 may be configured to deflect radially inwards to facilitate distal advancement of the soft tissue anchor 120a through the frame 100, implant 12, and/or body tissue and resume an expanded outwardly extending configuration (as shown in FIG. 6B) after insertion, although this is not required. The distal fixation end 124 or distal tip of the soft tissue anchor 120a is configured to lead the soft tissue anchor 120a as it is advanced into the bodily tissue. The barbs 128 are angled or sloped such that the soft tissue anchor 120a is easily advanced distally through the tissue but proximal retraction of the soft tissue anchor 120a is difficult or precluded. This may help ensure that once the soft tissue anchor 120a is assembled with the frame 100 and/or implant 12 and/or is implanted into the tissue it is self-anchoring. The angle and/or length of the barbs 128 may be varied It is contemplated that the barbs 128 may have structures or shapes different from an angled surface, including, but not limited to, curves, steps, etc. It is contemplated that other structural features in place of or in addition to the barbs 128 may be used to help secure the soft tissue anchor 120a to the frame 100 and implant 12 and/or within the tissue.

The intermediate connecting portion or shank 126 may be an elongate shaft having a generally cylindrical shape; however, the intermediate connecting portion or shank 126 may take other shapes as desired. While the intermediate connecting portion or shank 126 is illustrated as having a generally uniform cross-sectional dimension, it is contemplated that the cross-sectional dimension of the intermediate connecting portion or shank 126 may vary along a length thereof. For example, the intermediate connecting portion or shank 126 may decrease in cross-sectional dimension from the proximal head portion 122 towards the distal fixation end 124. This is just one example. Other configurations may be used, as desired. It is contemplated that the soft tissue anchor 120a may be formed as a single monolithic structure or a series of components affixed to one another, as desired.

It is further contemplated that the soft tissue anchor 120a may take other configurations. For example, the soft tissue anchor 120a may be a barbed “U” shaped tissue anchor similar to a staple, having two distally extending legs. In such an instance, the barbed edges or legs penetrate and secure within the tissue while the connecting portion or bridge of the “U” provides a buttress to hold down the implant 12 between the barbed legs. In other cases, the soft tissue anchor 120a may be a screw having a threaded shank. The threaded region may penetrate the tissue while the head of the screw provides a buttress to hold down the implant 12. These are just some examples.

The attachment arm 110a may include a recess 114a to accommodate the proximal head portion 122 therein. For example, the recess 114a may be a generally circular recess configured to receive the circular proximal head portion 122 of the soft tissue anchor 120. However, the recess 114a may take other shapes, as desired. In some cases, the recess 114a may extend partially through a thickness 116a of the attachment arm 110a to define a region 118a of the attachment arm 110a having a reduced thickness. The recess 114a may be sized and shaped to receive the proximal head portion 122 of the soft tissue anchor 120a therein. The proximal head portion 122 of the soft tissue anchor 120a may be recessed or inset into the recess 114a. For example, the recess 114a may be deep enough that the proximal head portion 122 is level with or lower than the first surface 106 (e.g., surface facing away from the implant 12) of the frame 100. This may help reduce a delivery profile of the frame 100. However, this is not required. In some cases, the proximal head portion 122 of the soft tissue anchor 120a may extend above or beyond the first surface 106 of the frame 100.

In some cases, a through hole or aperture 140a may be formed through the reduced thickness region 118a. The through hole 140a may be sized and shaped to receive the intermediate connecting portion 126 and allow the distal fixation end 124 to pass therethrough beyond the lower side 108 of the frame 100. In some embodiments, the through hole 140a may be pre-formed in the attachment arm 110a. In other embodiments, the through hole 140a may be formed as the soft tissue anchor 120a is assembled with the attachment arm 110a. For example, the reduced thickness region 118a may be configured to be punctured or fracture as the distal fixation end 124 is pushed through the reduced thickness region 118a. It is contemplated that the implant 12 may be positioned along the frame 100 such that as the soft tissue anchor 120a is assembled with the attachment arm 110a, the distal fixation end 124 pierces and penetrates through the implant 12 such that the implant 12 is coupled to the frame 100, such as between the lower side 108 of the frame and the barbed distal tip of the soft tissue anchor 120a, as shown in FIG. 5 and FIGS. 6A-6B.

The frame 100, implant 12 and soft tissue anchor 120a may be delivered to the body using a delivery system and method similar to that described above with respect to FIGS. 2-4. Once the implant 12 is positioned at the target location, it may be temporarily secured, using, for example, a bone tack associated with the delivery frame 100 of the delivery device. The delivery shaft of the delivery device may be released from the frame 100 and retracted from the portal to allow other devices to be inserted. In some cases, a tether may be coupled to the frame 100 and extend out of the body to facilitate retrieval of the frame 100 once it is uncoupled from the implant 12. A tool may then be advanced to decouple the soft tissue anchor 120a from the frame 100. In some cases, the tool may be similar to a punch type tool, although this is not required. The tool may be aligned with the proximal head portion 122 of the soft tissue anchor 120a. A force may then be applied to the proximal head portion 122 by the tool to distally advance the soft tissue anchor 120a such that the distal fixation end 124 pierces the tissue to penetrate into tissue. It is contemplated that the reduced thickness region 118a of the attachment arm 110a may be configured to be frangible such that the reduced thickness region 118a breaks away from the attachment arm 110a as the force is applied to the proximal head portion 122 to pass the proximal head portion 122 through the through hole or aperture 140a. In some cases, the reduced thickness region 118a may break away entirely such that the reduced thickness region 118a remains in the body with the soft tissue anchor 120a and implant 12. In other cases, the reduced thickness region 118a may be configured to deform as the proximal head portion 122 passes through the through hole or aperture 140a such that the reduced thickness region 118a remains connected to the attachment arm 110a. The tool may be repositioned to align with another soft tissue anchor 120 coupling the implant 12 with the frame 100, and a force may be exerted on the soft tissue anchor 120 with the tool to decouple the additional soft tissue anchor 120 from the frame 100 and penetrate into underlying body tissue (e.g., tendon). This may be repeated for each soft tissue anchor 120 provided with the frame 100. This may allow multiple soft tissue anchors 120 to be deployed without requiring the clinician to individually load each soft tissue anchor 120 into the tool and to repeatedly insert and remove the tool from the incision accessing the treatment site.

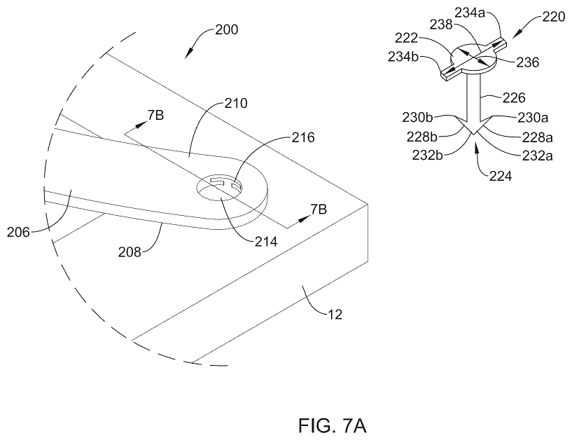

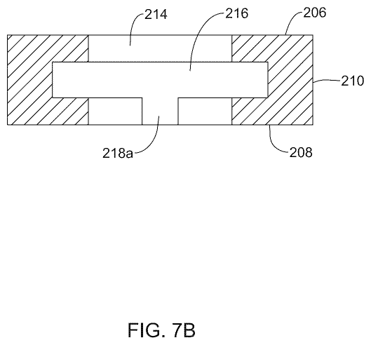

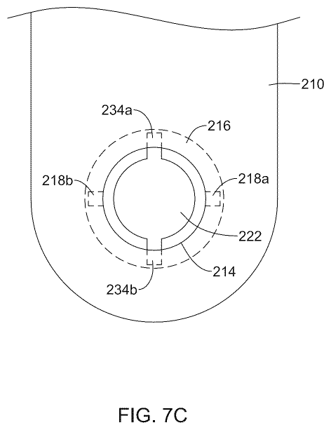

FIG. 7A illustrates an exploded perspective view of an illustrative attachment arm 210 of a frame 200 and soft tissue anchor 220. The frame 200 may be similar in form and function to the frame 100 described herein. The soft tissue anchor 220 may be configured to be assembled with the frame 200 and an implant 12 to releasably secure the implant to the frame 200 during delivery of the implant 12 to a target site. FIG. 7B is a cross-sectional view of the illustrative attachment arm 210, taken at line 7B-7B of FIG. 7A. FIG. 7C is a top view of the soft tissue anchor 220 assembled with the illustrative attachment arm 210. While the soft tissue anchor 220 is described with respect to a single attachment arm 210, it should be understood a soft tissue anchor 220 may be provided in any number of attachment arms 210, as desired.

The soft tissue anchor 220 may include a proximal head portion 222 configured to hold down the implant 12 and a distal fixation end 224 configured to penetrate the tissue. An elongate intermediate connecting portion or shank 226 may extend between the proximal head portion 222 and the distal fixation end 224. It is contemplated that the proximal head portion 222 may be a generally planar component having a width greater than a width of the intermediate connecting portion or shank 226. In some cases, the proximal head portion 222 may be generally disc-shaped having a generally circular cross-sectional shape. However, this is not required. The proximal head portion 222 may have other cross-sectional shapes, such as, but not limited, square, rectangular, elliptical, polygonal, etc. The proximal head portion 222 may include one or more radially extending tabs 234a, 234b (collectively, 234) extending from an outer perimeter thereof such that the tabs 234 define a region of the proximal head portion 222 having a greater cross-sectional dimension 238 than a cross-sectional dimension 236 of the proximal head portion free from the tabs 234. The tabs 234 may be sized and shaped to mate with a corresponding slot and/or groove in the attachment arm 210, as will be described in more detail herein. In some instances, the one or more tabs 234 may be helically arranged around the proximal head portion 222 like male threading.

The distal fixation end 224 may be generally “V” shaped or pointed, and may include a first barb 228a and a second barb 228b (collectively, 228). The distal fixation end 224 may be shaped such that proximal retraction of the soft tissue anchor 220 is prevented or hindered once the distal fixation end 224 is assembled with the frame 200 and/or body tissue. For example, the barbs 228 may each include a proximal end 230a, 230b (collectively, 230) radially spaced from the intermediate connecting portion or shank 226 and a distal end 232a, 232b (collectively, 232) in contact with or coupled to the distal fixation end 224. In some cases, fewer than two or more than two barbs 228 may be provided. In some cases, the proximal ends 230 of the barbs 228 may be configured to deflect radially inwards to facilitate distal advancement of the soft tissue anchor 220 through the frame 200, implant 12, and/or body tissue and resume an expanded outwardly extending configuration (as shown in FIG. 7A) after insertion, although this is not required. The distal fixation end 224 or distal tip of the soft tissue anchor 220 is configured to lead the soft tissue anchor 220 as it is advanced into the bodily tissue. The barbs 228 are angled or sloped such that the soft tissue anchor 220 is easily distally advanced through the tissue but proximal retraction of the soft tissue anchor 220 is difficult or precluded. This may help ensure that once the soft tissue anchor 220 is assembled with the frame 200 and/or implant 12 and/or is implanted into the tissue it is self-anchoring. The angle and/or length of the barbs 228 may be varied It is contemplated that the barbs 228 may have structures or shapes different from an angled surface, including, but not limited to, curves, steps, etc. It is contemplated that other structural features in place of or in addition to the barbs 228 may be used to help secure the soft tissue anchor 220 to the frame 200 and implant 12 and/or within the tissue.

The intermediate connecting portion or shank 226 may be an elongate rod having a generally cylindrical shape; however, the intermediate connecting portion or shank 226 may take other shapes as desired. While the intermediate connecting portion or shank 226 is illustrated as having a generally uniform cross-sectional dimension, it is contemplated that the cross-sectional dimension of the intermediate connecting portion or shank 226 may vary along a length thereof. For example, the intermediate connecting portion or shank 226 may decrease in cross-sectional dimension from the proximal head portion 222 towards the distal fixation end 224. This is just one example. Other configurations may be used, as desired. It is contemplated that the soft tissue anchor 220 may be formed as a single monolithic structure or a series of components affixed to one another, as desired.

It is further contemplated that the soft tissue anchor 220 may take other configurations. For example, the soft tissue anchor 220 may be a screw having a threaded distal end region. The threaded region may penetrate the tissue while the head of the screw provides a buttress to hold down the implant. This is just an example.

The attachment arm 210 may include a generally circular aperture 214 configured to receive the proximal head portion 222 of the soft tissue anchor 220. The aperture 214 may extend through a thickness of the attachment arm 210 from a first surface 206 that faces away from the implant 12 when the implant 12 is attached to the frame 200 (e.g., a first surface that faces away from a target site in the body) and a second surface 208 that faces or is juxtaposed with the example implant 12 (e.g., a second surface that faces a target site in the body). The aperture 214 may be sized and shaped to receive the proximal head portion 222 of the soft tissue anchor 220 therein. The aperture 214 may include a circumferential groove 216 extending about a perimeter thereof and positioned between the first surface 206 and the second surface 208. The groove 216 may have a diameter that is greater than a diameter of the aperture 214. It is further contemplated that the groove 216 may have a diameter that is at least as great as the major cross-sectional dimension 238 of the proximal head portion 222 of the soft tissue anchor 220. As will be described in more detail, this may allow the tab(s) 234 of the proximal head portion 222 to be rotatably disposed within the groove 216. In some instances, the aperture 214 may further include a first axially extending slot 218a and/or a second axially extending slot 218b (see, for example, FIG. 7C) (collectively, 218). The slots 218 may extend from the circumferential groove 216 to the second surface 208, as shown in FIG. 7B. The slots 218 may have a width that is greater than a width of the tabs 234 to allow the tabs 234 to slide therein. It is contemplated that a depth of the slots 218 may be similar to a depth of the groove 216, although this is not required. Said differently the slots 218 may extend to a same radial position as the circumferential groove 216. The slots 218 may be positioned on diametrically opposite sides of the aperture 214 (i.e., circumferentially spaced 180 degrees apart), for example. In other instances, the circumferential groove 216 may be a helical groove extending to the second surface 208 of the frame 200.

To assemble the soft tissue anchor 220 with the attachment arm 210, the tabs 234 of the proximal head portion 222 may be aligned with the axially extending slots 218 adjacent the second surface 208 of the attachment arm 210. The tabs 234 may then be advanced within the slots 218 towards the circumferential groove 216 until further movement of the tabs 234 is precluded by an upper edge of the circumferential groove 216. The soft tissue anchor 220 may then be rotated such that the tabs 234 are circumferentially displaced from the slots 218, as shown in FIG. 7C. Once the tabs 234 have been circumferentially displaced from the slots 218 axial movement (e.g., movement between the first and second surfaces 206, 208) of the soft tissue anchor 220 may be precluded. In instances in which the circumferential groove 216 is a helical groove, the tabs 234 may enter the circumferential groove 216 at the second surface 208 and be rotated therein to position the tabs 234 in the circumferential groove 216. The implant 12 may be affixed adjacent to the second surface 208 of the frame 200 after assembly of the soft tissue anchors 220 therewith. It is contemplated that the soft tissue anchors 220 may penetrate or pierce the implant 12 (similar to FIG. 6B) to secure the implant 12 to the frame 200.

The frame 200, implant 12, and soft tissue anchor 220 may be delivered to the body using a delivery system and method similar to that described above with respect to FIGS. 2-4. Once the implant 12 is positioned at the target location, it may be temporarily secured, using, for example, a bone tack. The delivery system may be released and retracted from the portal to allow other devices to be inserted. In some cases, a tether may be coupled to the frame 200 and extend out of the body to facilitate retrieval of the frame 200 once it is uncoupled from the implant 12. A tool may then be advanced to decouple the soft tissue anchor 220 from the frame 200. In some cases, the tool may be similar to a screw-driver type tool, although this is not required. In some cases, the tool may include protrusions or recesses configured to engage the tabs 234 to facilitate rotation of the soft tissue anchor 220. In other embodiments, the tool may be configured to engage a feature in the proximal head portion 222, such as, but not limited to, grooves, recesses, angled sides, etc. The tool may be aligned and engaged with the proximal head portion 222 of the soft tissue anchor 220. A rotational force may then be applied to the tool to rotate the soft tissue anchor 220 until the tabs 234 align with the slots 218. In some cases, an axial force may be applied while rotating the soft tissue anchor 220 such that when the tabs 234 align with the slots 218 the soft tissue anchor 220 is axially displaced within the aperture 214. If an axial force is not applied during rotation, once the tabs 234 align with the slots 218 an axial force may be applied to the soft tissue anchor 220 to advance the tabs 234 along the slots 218 towards the second surface 208. The soft tissue anchor 220 may be axially displaced until the proximal head portion 222 exits the aperture 214 and the distal fixation end 224 pierces the body tissue. In some instances, after the soft tissue anchor 220 has been rotated by the tool to align the tabs 234 with the slots 218, the tool may then be used as a punch or driver to exert the axial force on the soft tissue anchor 220, such as being hit with a mallet to drive the soft tissue anchor 220 into the tissue. In instances in which the circumferential groove 216 is a helical groove, rotation of the soft tissue anchor 220 by the tool may screw the soft tissue anchor 220 into the tissue. The tool may be repositioned to align with another soft tissue anchor 220 and the steps repeated to decouple the additional soft tissue anchor 220 from the frame 200. This may be repeated for each soft tissue anchor 220. This may allow multiple soft tissue anchor 220 to be deployed without requiring the clinician to individually load each soft tissue anchor 220 into the tool and to repeatedly insert and remove the tool.

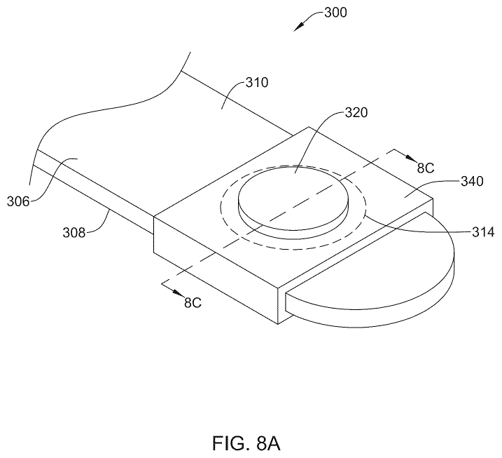

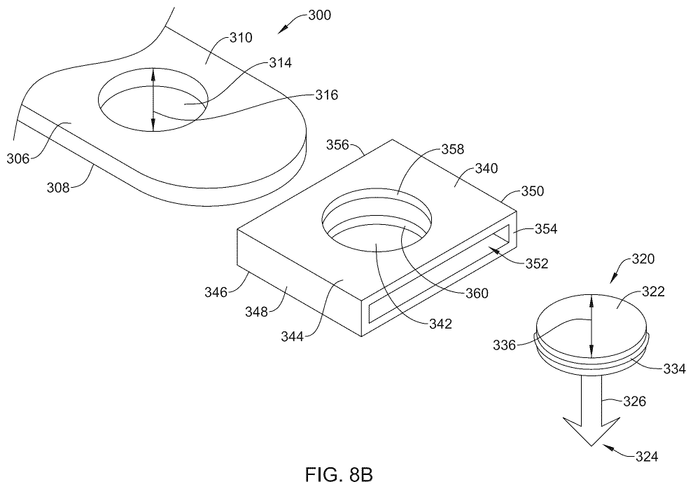

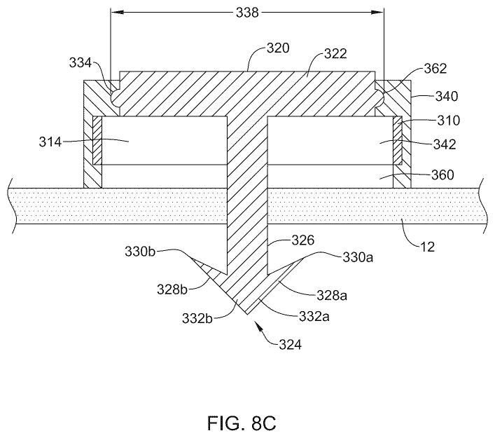

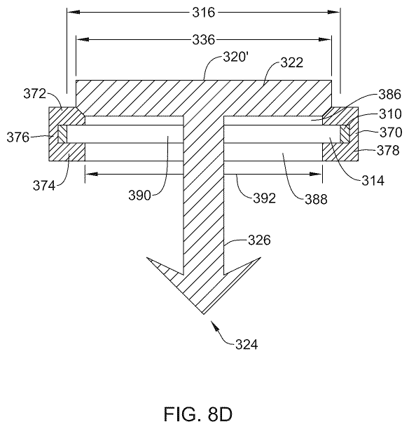

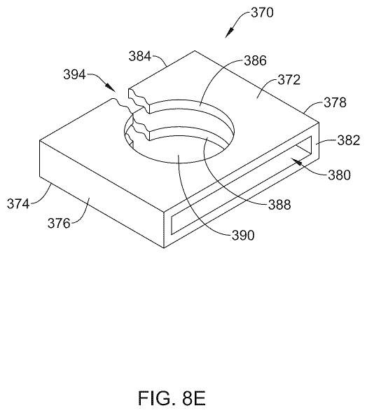

FIG. 8A illustrates a perspective view of another illustrative attachment arm 310 of a frame 300 and soft tissue anchor 320. FIG. 8B is an exploded perspective view of the illustrative attachment arm 310 of a frame 300 and soft tissue anchor 320 of FIG. 8A. The frame 300 may be similar in form and function to the frame 100 described herein. The soft tissue anchor 320 may be configured to be assembled with the frame 300 and an implant 12 to releasably secure the implant to the frame 300. FIG. 8C is a cross-sectional view of the illustrative attachment arm 310, taken at line 8C-8C of FIG. 8A. While the soft tissue anchor 320 is described with respect to a single attachment arm 310, it should be understood a soft tissue anchor 320 may be provided in any number of attachment arms 310, as desired.

In the illustrative embodiment of FIGS. 8A-8C, the soft tissue anchor 320 may be removably coupled to the attachment arm 310 via a retainer 340. The retainer 340 may be configured to be slid over the free end of the attachment arm 310. Generally, the soft tissue anchor 320 may be advanced through an aperture 342 in the retainer and an aperture 314 in the attachment arm 310. The retainer 340 may include features configured to engage a mating feature of the soft tissue anchor 320 to releasably secure the soft tissue anchor 320 thereto.

The soft tissue anchor 320 may include a proximal head portion 322 configured to hold down the implant 12 and a distal fixation end 324 configured to penetrate the tissue. An elongate intermediate connecting portion or shank 326 may extend between the proximal head portion 322 and the distal fixation end 324. It is contemplated that the proximal head portion 322 may be a generally planar component having a width greater than a width of the intermediate connecting portion or shank 326. In some cases, the proximal head portion 322 may be generally disc-shaped having a generally circular cross-sectional shape. However, this is not required. The proximal head portion 322 may have other cross-sectional shapes, such as, but not limited, square, rectangular, elliptical, polygonal, etc. The proximal head portion 322 may include a radially extending rim or detent 334 extending from a side surface of the proximal head portion 322. In some cases, the rim or detent 334 may extend about an entire circumference of the proximal head portion 322. In other embodiments, the detent 334 may include a plurality of detents (e.g., two, three, four, or more) spaced about the circumference of the proximal head portion 322. When more than one detent 334 is provided, the detents 334 may be protuberances uniformly spaced or eccentrically spaced, as desired. The detent 334 may be sized and shaped to mate with a corresponding slot and/or groove in the retainer 340, as will be described in more detail herein.

The distal fixation end 324 may be generally “V” shaped or pointed, and may include a first barb 328a and a second barb 328b (collectively, 328). The distal fixation end 324 may be shaped such that proximal retraction of the soft tissue anchor 320 is prevented or hindered once the distal fixation end 324 is assembled with the frame 300 and/or body tissue. For example, the barbs 328 may each include a proximal end 330a, 330b (collectively, 330) radially spaced from the intermediate connecting portion 326 and a distal end 332a, 332b (collectively, 332) in contact with or coupled to the distal fixation end 324. In some cases, fewer than two or more than two barbs 328 may be provided. In some cases, the proximal ends 330 of the barbs 328 may be configured to deflect radially inwards to facilitate distal advancement of the soft tissue anchor 320 through the frame 300, implant 12, and/or body tissue and resume an expanded outwardly extending configuration (as shown in FIGS. 8B and 8C) after insertion, although this is not required. The distal fixation end 324 or distal tip of the soft tissue anchor 320 is configured to lead the soft tissue anchor 320 as it is advanced into the bodily tissue. The barbs 328 are angled or sloped such that the soft tissue anchor 320 is easily distally advanced through the tissue but proximal retraction of the soft tissue anchor 320 is difficult or precluded. This may help ensure that once the soft tissue anchor 320 is assembled with the frame 300 and/or implant 12 and/or is implanted into the tissue it is self-anchoring. The angle and/or length of the barbs 328 may be varied It is contemplated that the barbs 328 may have structures or shapes different from an angled surface, including, but not limited to, curves, steps, etc. It is contemplated that other structural features in place of or in addition to the barbs 328 may be used to help secure the soft tissue anchor 320 to the frame 300 and implant 12 and/or within the tissue.

The intermediate connecting portion or shank 326 may be an elongate rod having a generally cylindrical shape; however, the intermediate connecting portion or shank 326 may take other shapes as desired. While the intermediate connecting portion or shank 326 is illustrated as having a generally uniform cross-sectional dimension, it is contemplated that the cross-sectional dimension of the intermediate connecting portion or shank 326 may vary along a length thereof. For example, the intermediate connecting portion or shank 326 may decrease in cross-sectional dimension from the proximal head portion 322 towards the distal fixation end 324. This is just one example. Other configurations may be used, as desired. It is contemplated that the soft tissue anchor 320 may be formed as a single monolithic structure or a series of components affixed to one another, as desired.

It is further contemplated that the soft tissue anchor 320 may take other configurations. For example, the soft tissue anchor 320 may be a barbed “U” shaped tissue anchor similar to a staple, having two distally extending legs. In such an instance, the barbed edges or legs penetrate and secure within the tissue while the connecting portion or bridge of the “U” provides a buttress to hold down the implant 12 between the barbed legs. In other embodiments, the soft tissue anchor 320 may be a screw having a threaded distal end region. The threaded region may penetrate the tissue while the head of the screw provides a buttress to hold down the implant. This is just an example.

The attachment arm 310 may include a generally circular aperture 314 configured to receive a portion of the soft tissue anchor 320. However, the aperture 314 may take other shapes, as desired. The aperture 314 may have a cross-sectional shape similar to the general cross-sectional shape of the proximal head portion 322 of the soft tissue anchor 320 and extend through a thickness of the attachment arm 310 from a first surface 306 that faces away from the implant 12 when the implant 12 is attached to the frame 300 (e.g., a first surface that faces away from a target site in the body) and a second surface 308 that faces or is juxtaposed with the example implant 12 (e.g., a second surface that faces a target site in the body). The aperture 314 may be sized and shaped to allow the proximal head portion 322 of the soft tissue anchor 320 to pass therethrough. For example, the aperture 314 may have a diameter 316 that is greater than a diameter 336 of the proximal head portion 322 and greater than a maximum diameter 338 of the proximal head portion 322 (e.g., at the detent(s) 334).

The retainer 340 may include a top side 344 that faces away from the implant 12 when the implant 12 is attached to the frame 300, a bottom side 346 that faces the implant 12 when the implant 12 is attached to the frame 300, a first lateral side 348 extending between the top side 344 and the bottom side 346, and a second lateral side 350 opposite the first lateral side and extending between the top side 344 and the bottom side 346. Collectively, the top side 344, bottom side 346, first lateral side 348, and second lateral side 350 may define a channel 352 that extends from a first end 354 to a second end 356 of the retainer 340. The channel 352 may be sized and shaped to allow the retainer 340 to be slid over the attachment arm 310 (as shown in FIG. 8A) while preventing the retainer 340 from unintentionally disengaging from the attachment arm 310 prior to assembly with the soft tissue anchor 320. The retainer 340 may further include a first opening 358 in the top side 344 and a second opening 360 defined in the bottom side 346. The first and second openings 358, 360 may be generally circular; however, the first and second openings 358, 360 may take other shapes, as desired. The first and second openings 358, 360 may be generally aligned to define an aperture 342 extending from the top side 344 to the bottom side 346. The first and second openings 358, 360 may have similar or the same diameters or may have differing diameters, as desired. In some embodiments, the first opening 358 may have a diameter that is similar to or the same as the diameter 336 of the proximal head portion 322 of the soft tissue anchor 320 while the second opening 360 may have a diameter that is greater than the maximum diameter 338 of the proximal head portion 322 at the detent 334.

To assemble the soft tissue anchor 320 with the attachment arm 310, the retainer 340 may be advanced over the free end of the attachment arm 310 until the aperture 342 of the retainer 340 is generally aligned with the aperture 314 of the attachment arm 310. The distal fixation end 324 of the soft tissue anchor 320 may then be advanced through the apertures 314, 342 in a direction from the top side 344 towards the bottom side 346 of the retainer 340, or from the bottom side 346 toward the top side 344, if desired. The soft tissue anchor 320 may be advanced until the detent(s) 334 on the proximal head region 322 engage a mating recess 362 (see, for example, FIG. 8C) formed in a side wall of the first opening 358 formed in the top side 344 of the retainer 340. The detent 334 and corresponding recess 362 may from a snap fit or other mechanical engagement to maintain the soft tissue anchor 320 in a desired configuration during delivery of the implant 12. It is contemplated that the implant 12 may be positioned adjacent to the second surface 308 of the frame 300 and/or the bottom side 346 of the retainer 340 during assembly such that the soft tissue anchor 320 pierces the implant 12 and secures it to the frame 300.

The frame 300, implant 12, and soft tissue anchor 320 may be delivered to the body using a delivery system and method similar to that described above with respect to FIGS. 2-4. Once the implant 12 is positioned at the target location, it may be temporarily secured, using, for example, a bone tack. The delivery system may be released and retracted from the portal to allow other devices to be inserted. In some cases, a tether may be coupled to the frame 300 and extend out of the body to facilitate retrieval of the frame 300 once it is uncoupled from the implant 12. A tool may then be advanced to decouple the soft tissue anchor 320 from the frame 300. In some cases, the tool may be similar to a punch type tool, although this is not required. The tool may be aligned with the proximal head portion 322 of the soft tissue anchor 320. A force may then be applied, such as with a mallet, to distally advance the soft tissue anchor 320 such that the distal fixation end 324 or distal tip pierces the tissue. It is contemplated that the top side 344 of the retainer 340 may flex, deflect, deform, or fracture as the force is applied to the proximal head portion 322 allowing the detent(s) 334 to release from the mating recess 362. As noted above, the second opening 360 may be sized such that the maximum diameter 338 of the proximal head portion 322 easily passes therethrough, although this is not required. The tool may be repositioned to align with another soft tissue anchor 320 and a force exerted on the soft tissue anchor 320 to decouple it from the frame 300. This may be repeated for each soft tissue anchor 320. This may allow multiple soft tissue anchor 320 to be deployed without requiring the clinician to individually load each soft tissue anchor 320 into the tool and to repeatedly insert and remove the tool.

FIG. 8D illustrates a cross-sectional view of an alternative retainer 370 assembled with the attachment arm 310 and soft tissue anchor 320′. For clarity, an implant is not illustrated. However, it should be understood an implant 12 may be assembled with the attachment arm 310, soft tissue anchor 320′, and retainer 370 in a manner similar to that illustrated in FIG. 8C. FIG. 8E is a perspective view of the illustrative retainer 370 after a soft tissue anchor 320′ has been deployed therefrom. The soft tissue anchor 320′ may be similar in form and function to the soft tissue anchor 320 described with respect to FIGS. 8A-8C and like elements are labeled with like numbers. The soft tissue anchor 320′ may be free from radially extending detents, although this is not required. In some instances, the lower surface of the proximal head portion 322 of the soft tissue anchor 320′ may include a beveled surface and/or the upper surface or top side 372 of the retainer 370 may include a beveled surface leading into the first opening 386 of the retainer 370 to facilitate advancement of the head portion 322 of the soft tissue anchor 320′ through the first opening 386.

The retainer 370 may include a top side 372 that faces away from the implant 12 when the implant 12 is attached to the frame 300, a bottom side 374 that faces the implant 12 when the implant 12 is attached to the frame 300, a first lateral side 376 extending between the top side 372 and the bottom side 374, and a second lateral side 378 opposite the first lateral side and extending between the top side 372 and the bottom side 374. Collectively, the top side 372, bottom side 374, first lateral side 376, and second lateral side 378 may define a channel 380 that extends from a first end 382 to a second end 384 of the retainer 370. The channel 380 may be sized and shaped to allow the retainer 370 to be slid over the attachment arm 310 while preventing the retainer 370 from unintentionally disengaging from the attachment arm 310 prior to assembly with the soft tissue anchor 320′. The retainer 370 may further include a first opening 386 in the top side 372 and a second opening 388 defined in the bottom side 374. The first and second openings 386, 388 may be generally circular; however, the first and second openings 386, 388 may take other shapes, as desired. The first and second openings 386, 388 may be generally aligned to define an aperture 390 extending from the top side 372 to the bottom side 374. The first and second openings 386, 388 may have a similar or the same diameters 392, however this is not required. It is contemplated the first and second openings 386, 388 may have differing diameters, as desired. The diameter 392 of the openings 386, 388 may be less than the diameter 336 of the proximal head portion 323 of the soft tissue anchor 320′ such that the proximal head portion 322 rests on top of the top side 372, as shown in FIG. 8D, when the soft tissue anchor 320′ is assembled with the retainer 370 and the attachment arm 310. The diameter 316 of the aperture 314 of the attachment arm 310 may be greater than the diameter 336 of the proximal head portion 322 (and the diameter 392 of the openings 386, 388) such that the proximal head portion 322 of the soft tissue anchor 320′ passes freely through the aperture 314 at deployment.

To assemble the soft tissue anchor 320′ with the attachment arm 310, the retainer 370 may be advanced over the free end of the attachment arm 310 until the aperture 390 of the retainer 370 is generally aligned with the aperture 314 of the attachment arm 310. The distal fixation end 324 of the soft tissue anchor 320′ may then be advanced through the apertures 314, 390 in a direction from the top side 372 towards the bottom side 374 of the retainer 370. The soft tissue anchor 320′ may be advanced until a bottom surface of the proximal head region 322 engages the top side 372 of the retainer 370. It is contemplated that the distal fixation end 324 or distal tip of the soft tissue anchor 320′ may pierce the implant to secure the soft tissue anchor 320′ to the frame 300. For example, the structure of the distal fixation end 324 of the soft tissue anchor 320′ may prevent retraction of the anchor 320′ once it has pierced the implant. It is contemplated that the implant 12 may be positioned adjacent to the second surface 308 of the frame 300 and/or the bottom side 374 of the retainer 370 during assembly such that the soft tissue anchor 320′ pierces the implant 12 and secures it to the frame 300 during delivery of the implant 12 to the target site.

The frame 300, implant, and soft tissue anchor 320′ may be delivered to the body using a delivery system and method similar to that described above with respect to FIGS. 2-4. Once the implant is positioned at the target location, it may be temporarily secured, using, for example, a bone tack. The delivery system may be released and retracted from the portal to allow other devices to be inserted. In some cases, a tether may be coupled to the frame 300 and extend out of the body to facilitate retrieval of the frame 300 once it is uncoupled from the implant 12. A tool may then be advanced to decouple the soft tissue anchor 320′ from the frame 300. In some cases, the tool may be similar to a punch type tool, although this is not required. The tool may be aligned with the proximal head portion 322 of the soft tissue anchor 320′. A force may then be applied to distally advance the soft tissue anchor 320′ such that the distal fixation end 324 or distal tip pierces the tissue. It is contemplated that the top side 372 of the retainer 370 may flex, deflect, deform, or fracture as the force is applied to the proximal head portion 322 allowing the proximal head portion 322 to pass through the aperture 390. In some cases, a region of weakness may be formed in the retainer 370 such that the retainer 370 fractures at a predetermined location 394. However, this is not required. It is further contemplated that the retainer 370 may be flexible so as to allow the proximal head portion 322 to pass without mechanical damage to the retainer 370. In some cases, the second opening 388 may be sized such that the proximal head portion 322 easily passes therethrough, although this is not required. The tool may be repositioned to align with another soft tissue anchor 320′ and a force exerted on the soft tissue anchor 320′ to decouple it from the frame 300. This may be repeated for each soft tissue anchor 320′. This may allow multiple soft tissue anchors 320′ to be deployed without requiring the clinician to individually load each soft tissue anchor 320′ into the tool and to repeatedly insert and remove the tool.

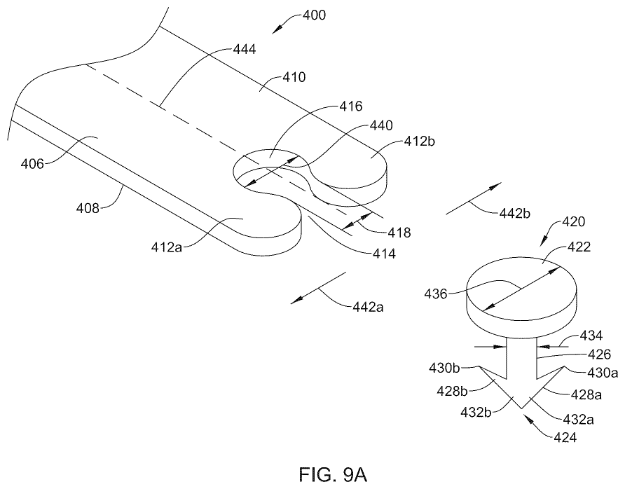

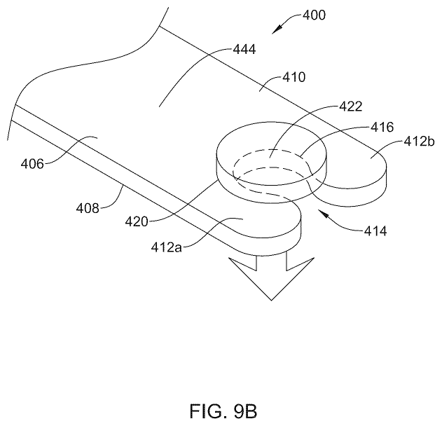

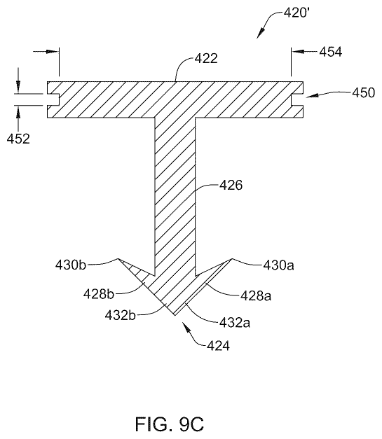

FIG. 9A illustrates an exploded perspective view of another illustrative attachment arm 410 of a frame 400 and soft tissue anchor 420. FIG. 9B is a perspective view of the illustrative attachment arm 410 of a frame 400 assembled with the soft tissue anchor 420. The frame 400 may be similar in form and function to the frame 100 described herein. The soft tissue anchor 420 may be configured to be assembled with the frame 400 and an implant 12 (not explicitly shown) to releasably secure the implant to the frame 400. While the soft tissue anchor 420 is described with respect to a single attachment arm 410, it should be understood a soft tissue anchor 420 may be provided in any number of attachment arms 410, as desired.

The soft tissue anchor 420 may include a proximal head portion 422 configured to hold down the implant 12 and a distal fixation end 424 configured to penetrate the tissue. An elongate intermediate connecting portion or shank 426 may extend between the proximal head portion 422 and the distal fixation end 424. It is contemplated that the proximal head portion 422 may be a generally planar component having a width greater than a width of the intermediate connecting portion 426. In some cases, the proximal head portion 422 may be generally disc-shaped having a generally circular cross-sectional shape. However, this is not required. The proximal head portion 422 may have other cross-sectional shapes, such as, but not limited, square, rectangular, elliptical, polygonal, etc.

The distal fixation end 424 may be generally “V” shaped or pointed, and may include a first barb 428a and a second barb 428b (collectively, 428). The distal fixation end 424 may be shaped such that proximal retraction of the soft tissue anchor 420 is prevented or hindered once the distal fixation end 424 is assembled with the frame 400 and/or body tissue. For example, the barbs 428 may each include a proximal end 430a, 430b (collectively, 430) radially spaced from the intermediate connecting portion or shank 426 and a distal end 432a, 432b (collectively, 432) in contact with or coupled to the distal fixation end 424. In some cases, fewer than two or more than two barbs 428 may be provided. In some cases, the proximal ends 430 of the barbs 428 may be configured to deflect radially inwards to facilitate distal advancement of the soft tissue anchor 420 through the frame 400, implant 12, and/or body tissue and resume an expanded outwardly extending configuration (as shown in FIG. 9A) after insertion, although this is not required. The distal fixation end 424 or distal tip of the soft tissue anchor 420 is configured to lead the soft tissue anchor 420 as it is advanced into the bodily tissue. The barbs 428 are angled or sloped such that the soft tissue anchor 420 is easily distally advanced through the tissue but proximal retraction of the soft tissue anchor 420 is difficult or precluded. This may help ensure that once the soft tissue anchor 420 is assembled with the frame 400 and/or implant 12 and/or is implanted into the tissue it is self-anchoring. The angle and/or length of the barbs 428 may be varied It is contemplated that the barbs 428 may have structures or shapes different from an angled surface, including, but not limited to, curves, steps, etc. It is contemplated that other structural features in place of or in addition to the barbs 428 may be used to help secure the soft tissue anchor 420 to the frame 400 and implant 12 and/or within the tissue.

The intermediate connecting portion or shank 426 may be an elongate rod having a generally cylindrical shape; however, the intermediate connecting portion or shank 426 may take other shapes as desired. While the intermediate connecting portion or shank 426 is illustrated as having a generally uniform diameter 434 cross-sectional dimension, it is contemplated that the cross-sectional dimension of the intermediate connecting portion or shank 426 may vary along a length thereof. For example, the intermediate connecting portion or shank 426 may decrease in cross-sectional dimension from the proximal head portion 422 towards the distal fixation end 424. This is just one example. Other configurations may be used, as desired. It is contemplated that the soft tissue anchor 420 may be formed as a single monolithic structure or a series of components affixed to one another, as desired.

It is further contemplated that the soft tissue anchor 420 may take other configurations. For example, the soft tissue anchor 420 may be a barbed “U” shaped tissue anchor similar to a staple, having two distally extending legs. In such an instance, the barbed edges or legs penetrate and secure within the tissue while the connecting portion or bridge of the “U” provides a buttress to hold down the implant 12 between the barbed legs. In other embodiments, the soft tissue anchor 420 may be a screw having a threaded distal end region. The threaded region may penetrate the tissue while the head of the screw provides a buttress to hold down the implant. This is just an example.

The attachment arm 410 may include a first flexible arm 412a and a second flexible arm 412b (collectively, 412) adjacent to a free end thereof. It is contemplated that the flexible arms 412 may be formed by removing some material from the attachment arm 410, or otherwise shaping the attachment arm 410 to include first and second flexible arms 412a/412b with a slot or opening therebetween, although other techniques may be used, as desired. The flexible arms 412 may increase in width towards the free end to define a distal opening 414 having a first width 418 and an aperture 416 having a second width 440. The width 418 of the distal opening 414 may be less than the width 440 of the aperture 416 spaced proximally of the distal opening 414. The distal opening 414 and the aperture 416 may extend through a thickness of the attachment arm 410 from a first surface 406 that faces away from the implant 12 when the implant 12 is attached to the frame 400 (e.g., a first surface that faces away from a target site in the body) and a second surface 408 that faces the example implant 12 (e.g., a second surface that faces a target site in the body). The aperture 416 may be sized and shaped to prevent passage of the proximal head portion 422 of the soft tissue anchor 420 therethrough. For example, the width 440 of the aperture 416 may be sufficient to accommodate the intermediate connecting portion or shank 426, yet less than a width or diameter 436 of the proximal head portion 422. The width of the opening 414 may be less than both the width or diameter 436 of the proximal head portion 422 and the width or diameter of the intermediate connecting portion or shank 426.

The flexible arms 412 may be configured to temporarily flex away from (or generally orthogonal to) a longitudinal axis 444 of the attachment arm 410, as shown at arrows 442a, 442b (collectively, 442). It is contemplated that the flexible arms 412 may be configured to flex in the directions shown at arrows 442 (i.e., flex away from one another) to temporarily widen the distal opening 414 in response to an applied force and to return to the original, unbiased, configuration in the absence of an applied force. In some cases, the intermediate connecting portion or shank 426 of the soft tissue anchor 420 may have a diameter 434 that is greater than the width 418 of the distal opening 414. To assemble the soft tissue anchor 420 with the attachment arm 410, the intermediate portion or shank 426 may be pushed through the distal opening 414 in a lateral direction parallel to the longitudinal axis 444 of the attachment arm 410. The intermediate portion or shank 426 may bias the flexible arms 412 outward 442 to allow the intermediate portion 426 to pass through the distal opening 414 and into the aperture 416. Once the intermediate portion or shank 426 has passed through the distal opening 414, the flexible arms 412 may return to or toward their original unbiased configuration (i.e., the flexible arms 412 may move toward one another). In the absence of an applied force, the intermediate portion or shank 426 is retained within the aperture 416 with the head portion 422 above the top surface 406 of the attachment arm 410 and the distal fixation end 424 located below the lower surface 408 of the attachment arm 410. The diameter 440 of the aperture 416 may be greater than the diameter 434 of the intermediate portion or shank 426 such that the intermediate portion 426 moves freely within the aperture 416. In other embodiments, the diameter 440 of the aperture 416 may be approximately the same as the diameter 434 of the intermediate portion or shank 426 to create a friction fit between the intermediate portion or shank 426 and the attachment arm 410. Alternatively, the soft tissue anchor 420 may be assembled with the attachment arm 410 by advancing the distal fixation end 424 axially through the aperture 416 in a direction from the first surface 406 to the second surface 408. It is contemplated that the barbs 428 may deflect towards the intermediate portion 426 to allow the distal fixation end 424 to pass through the aperture 416. The implant 12 may be affixed adjacent to the second surface 408 of the frame 400 simultaneously with or subsequent to the assembly of the soft tissue anchors 420 therewith. It is contemplated that the soft tissue anchors 420 may penetrate or pierce the implant 12 (similar to FIG. 6B) to secure the implant 12 to the frame 400 for delivery to a target site.

The frame 400, implant 12, and soft tissue anchor 420 may be delivered to the body using a delivery system and method similar to that described above with respect to FIGS. 2-4. Once the implant 12 is positioned at the target location, it may be temporarily secured, using, for example, a bone tack. The delivery system may be released and retracted from the portal to allow other devices to be inserted. In some cases, a tether may be coupled to the frame 400 and extend out of the body to facilitate retrieval of the frame 400 once it is uncoupled from the implant 12. A tool may then be advanced to decouple the soft tissue anchor 420 from the frame 400. In some cases, the tool may be similar to a punch type tool, although this is not required. The tool may be aligned with the proximal head portion 422 of the soft tissue anchor 420. A force may then be applied to distally advance the soft tissue anchor 420 such that the distal fixation end 424 or distal tip pierces the tissue. The tool may be repositioned to align with another soft tissue anchor 420 and a force exerted on the soft tissue anchor 420 to decouple it from the frame 400. This may be repeated for each soft tissue anchor 420. This may allow multiple soft tissue anchors 420 to be deployed without requiring the clinician to individually load each soft tissue anchor 420 into the tool and to repeatedly insert and remove the tool. Once each soft tissue anchor 420 has been driven into the tissue, the frame 400 may be retracted from the body. The retraction of the frame 400 may exert a lateral pulling force on the attachment arms 410 which in turn may cause the flexible arms 412 to bias outwards 442 allowing the attachment arms 410 to pass over the intermediate portion or shank 426 of the soft tissue anchors 420 releasing the frame 400 from the soft tissue anchors 420.

FIG. 9C illustrates a cross-sectional view of an alternative soft tissue anchor 420′ for use with the attachment arm 410 of FIGS. 9A and 9B. The soft tissue anchor 420′ may be similar in form and function to the soft tissue anchor 320 described with respect to FIGS. 9A-9B and like elements are labeled with like numbers. The soft tissue anchor 420′ may further include a circumferentially extending recess 450 formed in the proximal head portion 422. The recess 450 may have a height 452 that is similar to a thickness of the attachment arm 410 (e.g., the distance from the first surface 406 to the second surface 408). It is further contemplated that the cross-section or diameter 454 of the proximal head portion 422 at the recess 450 may be similar to, the same as, or greater than the diameter 440 of the aperture 416 in the attachment arm 410.

To assemble the soft tissue anchor 420′ with the attachment arm 410, the proximal head portion 422 may be pushed through the distal opening 414 in a lateral direction parallel to the longitudinal axis 444 of the attachment arm 410 with the recess 450 aligned with the attachment arm 410. The proximal head portion 422 may bias the flexible arms 412 outward (as shown in FIG. 9A) to allow the proximal head portion 422 to pass through the distal opening 414 and into the aperture 416. Once the proximal head portion 422 has passed through the distal opening 414, the flexible arms 412 return to or toward their original unbiased configuration or move laterally inwards to apply a gripping or pinching force on the proximal head portion 422. The recess 450 of the proximal head portion 422 may be positioned such that a portion of the flexible arms 412 are disposed within the recess 450. In the absence of an applied force, the proximal head portion 422 is retained within the aperture 416. The implant 12 may be affixed adjacent to the second surface 408 of the frame 400 simultaneously with or subsequent to the assembly of the soft tissue anchors 420′ therewith. It is contemplated that the soft tissue anchors 420′ may penetrate or pierce the implant 12 (similar to FIG. 6B) to secure the implant 12 to the frame 400.

The frame 400, implant 12, and soft tissue anchor 420′ may be delivered to the body using a delivery system and method similar to that described above with respect to FIGS. 2-4. Once the implant 12 is positioned at the target location, it may be temporarily secured, using, for example, a bone tack. The delivery system may be released and retracted from the portal to allow other devices to be inserted. In some cases, a tether may be coupled to the frame 400 and extend out of the body to facilitate retrieval of the frame 400 once it is uncoupled from the implant 12. A tool may then be advanced to decouple the soft tissue anchor 420′ from the frame 400. In some cases, the tool may be similar to a punch type tool, although this is not required. The tool may be aligned with the proximal head portion 422 of the soft tissue anchor 420′. A force may then be applied to distally advance the soft tissue anchor 420′ such that the distal fixation end 424 or distal tip pierces the tissue. The tool may be repositioned to align with another soft tissue anchor 420′ and a force exerted on the soft tissue anchor 420′ to decouple it from the frame 400. This may be repeated for each soft tissue anchor 420′. This may allow multiple soft tissue anchor 420′ to be deployed without requiring the clinician to individually load each soft tissue anchor 420′ into the tool and to repeatedly insert and remove the tool. Once each soft tissue anchor 420′ has been driven into the tissue, the frame 400 may be retracted from the body. The retraction of the frame 400 may exert a lateral pulling force on the attachment arms 410 which in turn may cause the flexible arms 412 to bias outwards 442 allowing the attachment arms 410 to pass over the proximal head portion 422 of the soft tissue anchors 420′ releasing the frame 400 from the soft tissue anchors 420′.

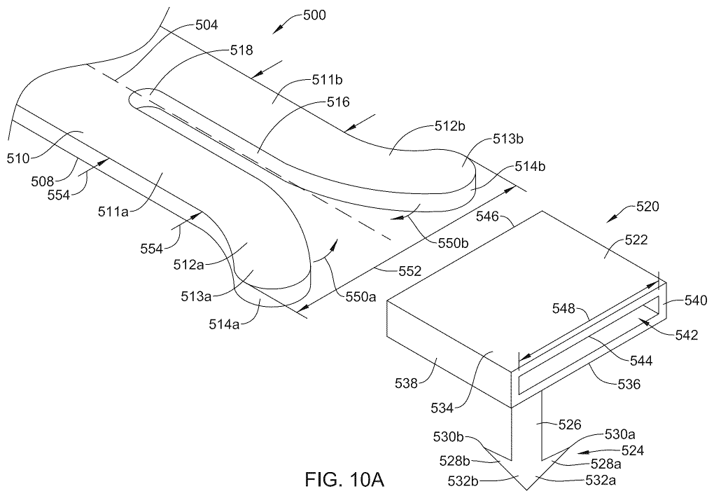

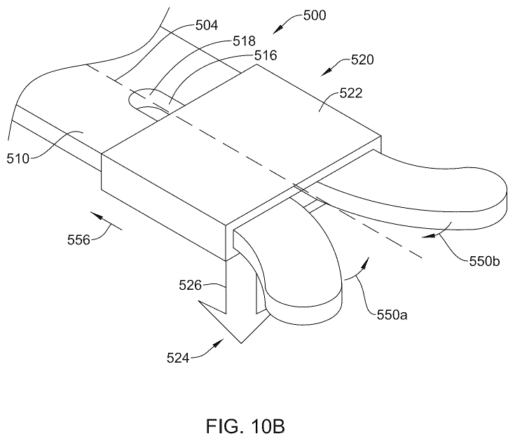

FIG. 10A illustrates an exploded perspective view of another illustrative attachment arm 510 of a frame 500 and soft tissue anchor 520. FIG. 10B is a perspective view of the illustrative attachment arm 510 of a frame 500 assembled with the soft tissue anchor 520. The frame 500 may be similar in form and function to the frame 100 described herein. The soft tissue anchor 520 may be configured to be assembled with the frame 500 and an implant 12 (not explicitly shown) to releasably secure the implant to the frame 500. While the soft tissue anchor 520 is described with respect to a single attachment arm 510, it should be understood a soft tissue anchor 520 may be provided in any number of attachment arms 510, as desired.

The soft tissue anchor 520 may include a proximal head portion 522 configured to hold down the implant 12 and a distal fixation end 524 configured to penetrate the tissue. An elongate intermediate connecting portion or shank 526 may extend between the proximal head portion 522 and the distal fixation end 524. It is contemplated that the proximal head portion 522 may have a width greater than a width of the intermediate connecting portion 526. The proximal head portion 522 may include a top side 534 that faces away from the implant 12 when the implant 12 is attached to the frame 500, a bottom side 536 that faces or is juxtaposed with the implant 12 when the implant 12 is attached to the frame 500, a first lateral side 538 extending between the top side 534 and the bottom side 536, and a second lateral side 540 opposite the first lateral side 538 and extending between the top side 534 and the bottom side 536. Collectively, the top side 534, bottom side 536, first lateral side 538, and second lateral side 540 may define a channel 542 that extends from a first end 544 to a second end 546 of the proximal head portion 522. The channel 542 may be sized and shaped to allow the proximal head portion 522 to be slid over the attachment arm 510 (as shown in FIG. 10B) while preventing the proximal head portion 522 from unintentionally disengaging from the attachment arm 510 prior to assembly with the soft tissue anchor 520.

The distal fixation end 524 may be generally “V” shaped or pointed, and may include a first barb 528a and a second barb 528b (collectively, 528). The distal fixation end 524 may be shaped such that proximal retraction of the soft tissue anchor 520 is prevented or hindered once the distal fixation end 524 is assembled with the frame 500 and/or body tissue. For example, the barbs 528 may each include a proximal end 530a, 530b (collectively, 530) radially spaced from the intermediate connecting portion or shank 526 and a distal end 532a, 532b (collectively, 532) in contact with or coupled to the distal fixation end 524. In some cases, fewer than two or more than two barbs 528 may be provided. In some cases, the proximal ends 530 of the barbs 528 may be configured to deflect radially inwards to facilitate distal advancement of the soft tissue anchor 520 through the frame 500, implant 12, and/or body tissue and resume an expanded outwardly extending configuration (as shown in FIG. 10A) after insertion, although this is not required. The distal fixation end 524 or distal tip of the soft tissue anchor 520 is configured to lead the soft tissue anchor 520 as it is advanced into the bodily tissue. The barbs 528 are angled or sloped such that the soft tissue anchor 520 is easily distally advanced through the tissue but proximal retraction of the soft tissue anchor 520 is difficult or precluded. This may help ensure that once the soft tissue anchor 520 is assembled with the frame 500 and/or implant 12 and/or is implanted into the tissue it is self-anchoring. The angle and/or length of the barbs 528 may be varied It is contemplated that the barbs 528 may have structures or shapes different from an angled surface, including, but not limited to, curves, steps, etc. It is contemplated that other structural features in place of or in addition to the barbs 528 may be used to help secure the soft tissue anchor 520 to the frame 500 and implant 12 and/or within the tissue.

The intermediate connecting portion or shank 526 may be an elongate rod having a generally cylindrical shape; however, the intermediate connecting portion or shank 526 may take other shapes as desired. While the intermediate connecting portion or shank 526 is illustrated as having a generally uniform diameter cross-sectional dimension, it is contemplated that the cross-sectional dimension of the intermediate connecting portion or shank 526 may vary along a length thereof. For example, the intermediate connecting portion or shank 526 may decrease in cross-sectional dimension from the proximal head portion 522 towards the distal fixation end 524. This is just one example. Other configurations may be used, as desired. It is contemplated that the soft tissue anchor 520 may be formed as a single monolithic structure or a series of components affixed to one another, as desired.

It is further contemplated that the soft tissue anchor 520 may take other configurations. For example, the soft tissue anchor 520 may be a barbed “U” shaped tissue anchor similar to a staple, having two distally extending legs. In such an instance, the barbed edges or legs penetrate and secure within the tissue while the connecting portion or bridge of the “U” provides a channel to engage the attachment arm 510 and provides a buttress to hold down the implant 12 between the barbed legs. In other embodiments, the soft tissue anchor 520 may be a screw having a threaded distal end region. The threaded region may penetrate the tissue while the head of the screw provides a buttress to hold down the implant. This is just an example.

The attachment arm 510 may include a first flexible arm 512a and a second flexible arm 512b (collectively, 512) adjacent to a free end thereof. It is contemplated that the flexible arms 512 may be formed by removing some material from the attachment arm 510, or otherwise shaping the attachment arm 510 to include first and second flexible arms 512a/512b with a slot or opening therebetween, although other techniques may be used, as desired. The flexible arms 512 may each include a proximal portion 511a, 511b (collectively, 511) which extends generally parallel to the longitudinal axis 504 of the attachment arm 510 and a distal portion 513a, 513b (collectively, 513) which extends at a non-parallel angle to the longitudinal axis 504 of the attachment arm 510. The arms 512 may define a gap 516 or a region free from material therebetween. A proximal end of the gap 516 may include a strain relief notch 518 in some instances. The gap 516 may allow the flexible arms 512 to flex inwards towards the longitudinal axis 504, as shown at arrows 550a, 550b (collectively, 550), and thus the flexible arms 512 may flex inwards toward one another. The free ends 514a, 514b (collectively, 514) of the flexible arms 512 may each curve away from the longitudinal axis 504 of the attachment arm 510 such that the width 552 of the attachment arm 510 adjacent the free ends 514 is greater than a width 554 adjacent to the proximal portion 511 of flexible arms and/or the attachment arm 510 adjacent the strain relief 518, for example. While the free ends 514 are described as having a generally curved configuration, it is contemplated that the free ends 514 may include other configurations which increases the width 552 at the free ends 514 relative to the width 554 adjacent to the strain relief 518.

The flexible arms 512 may be configured to temporarily flex towards (or generally orthogonal to) the longitudinal axis 504 of the attachment arm 510 and thus toward one another, as shown at arrows 550a, 550b (collectively, 550). It is contemplated that the flexible arms 512 may be configured to flex in the directions shown at arrows 550 to temporarily narrow the width 552 adjacent the free ends 514 in response to an applied force and to return to the original configuration in the absence of an applied force. The channel 542 of the proximal head portion 522 of the soft tissue anchor 520 may have a width 548 that is greater than the width 554 of the attachment arm 510 adjacent the strain relief 518 and less than the width 552 at the free ends 514 in their relaxed or unbiased configuration. Deflection of the flexible arms 512 in direction 550 may temporarily reduce the width 552 to allow the flexible arms 512 to be inserted into the channel 542 of the proximal head portion 522 of the soft tissue anchor 520. The soft tissue anchor 520 may be slid along the attachment arm 510 (or the attachment arm 510 slid within the channel 542) until the soft tissue anchor 520 is disposed over the proximal portions 511 of the flexible arms 512. Once the soft tissue anchor 520 is disposed over the proximal portions 511 of the flexible arms 512, the flexible arms 512 may return to or toward their original unbiased configuration, increasing the width 552 at the free ends 514. In the absence of an applied force, the distal portions 513 and the free ends 514 extend laterally beyond the width 548 of the channel 542 to retain the soft tissue anchor 520 on the attachment arm 510. The width 548 of the channel 542 may be greater than the width 554 of the proximal portion 511 of the flexible arms 512 so that the soft tissue anchor 520 may freely slide over that portion of the attachment arm 510. Alternatively, the width 548 of the channel 542 may be approximately the same as the width 554 of the proximal portion 511 of the flexible arms 512 to provide a friction fit and further secure the soft tissue anchor relative to the attachment arm 510. The implant 12 may be affixed adjacent to the second surface 508 of the frame 500 simultaneously with or subsequent to the assembly of the soft tissue anchors 520 therewith. It is contemplated that the soft tissue anchors 520 may penetrate or pierce the implant 12 (similar to FIG. 6B) to secure the implant 12 to the frame 500 for delivery to a target site.

The frame 500, implant 12, and soft tissue anchor 520 may be delivered to the body using a delivery system and method similar to that described above with respect to FIGS. 2-4. Once the implant 12 is positioned at the target location, it may be temporarily secured, using, for example, a bone tack. The delivery system may be released and retracted from the portal to allow other devices to be inserted. In some cases, a tether may be coupled to the frame 500 and extend out of the body to facilitate retrieval of the frame 500 once it is uncoupled from the implant 12. A tool may then be advanced to decouple the soft tissue anchor 520 from the frame 500. In some cases, the tool may be similar to a punch type tool, although this is not required. The tool may be aligned with the proximal head portion 522 of the soft tissue anchor 520. A force may then be applied to distally advance the soft tissue anchor 520 such that the distal fixation end 524 or distal tip pierces the tissue. The tool may be repositioned to align with another soft tissue anchor 520 and a force exerted on the soft tissue anchor 520 to decouple it from the frame 500. This may be repeated for each soft tissue anchor 520. This may allow multiple soft tissue anchors 520 to be deployed without requiring the clinician to individually load each soft tissue anchor 520 into the tool and to repeatedly insert and remove the tool. Once each soft tissue anchor 520 has been driven into the tissue, the frame 500 may be retracted from the body. The retraction of the frame 500 may exert a lateral pulling force on the attachment arms 510 which in turn may cause the flexible arms 512 to bias inwards 550 allowing the attachment arms 510 to pass through the channel 542 of the proximal head portion 522 of the soft tissue anchors 520 releasing the frame 500 from the soft tissue anchors 520.

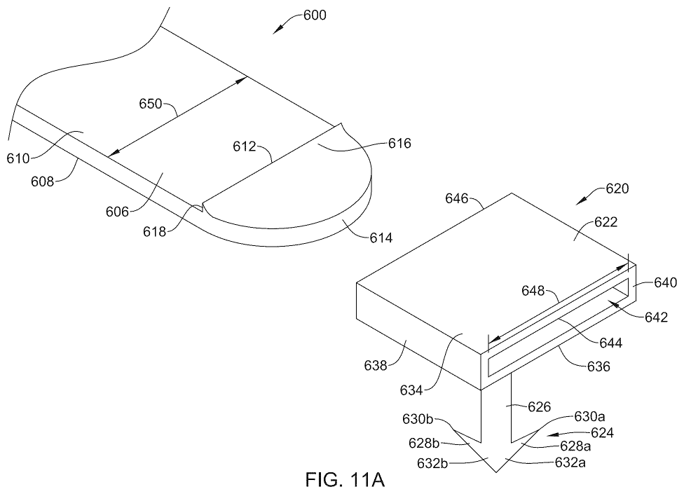

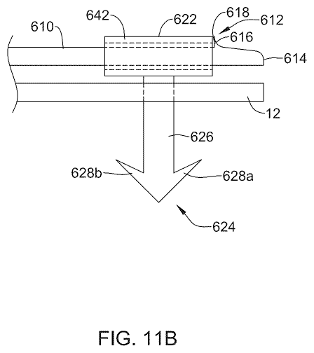

FIG. 11A illustrates an exploded perspective view of another illustrative attachment arm 610 of a frame 600 and soft tissue anchor 620. FIG. 11B is a side view of the illustrative attachment arm 610 of a frame 600 assembled with the soft tissue anchor 620 and an implant 12. The frame 600 may be similar in form and function to the frame 100 described herein. The soft tissue anchor 620 may be configured to be assembled with the frame 600 and an implant 12 (not explicitly shown) to releasably secure the implant 12 to the frame 600. While the soft tissue anchor 620 is described with respect to a single attachment arm 610, it should be understood a soft tissue anchor 620 may be provided in any number of attachment arms 610, as desired.

The soft tissue anchor 620 may include a proximal head portion 622 configured to hold down the implant 12 and a distal fixation end 624 configured to penetrate the tissue. An elongate intermediate connecting portion or shank 626 may extend between the proximal head portion 622 and the distal fixation end 624. It is contemplated that the proximal head portion 622 may have a width greater than a width of the intermediate connecting portion 626. The proximal head portion 622 may include a top side 634 that faces away from the implant 12 when the implant 12 is attached to the frame 600, a bottom side 636 that faces the implant 12 when the implant 12 is attached to the frame 600, a first lateral side 638 extending between the top side 634 and the bottom side 636, and a second lateral side 640 opposite the first lateral side 638 and extending between the top side 634 and the bottom side 636. Collectively, the top side 634, bottom side 636, first lateral side 638, and second lateral side 640 may define a channel 642 that extends from a first end 644 to a second end 646 of the proximal head portion 622. The channel 642 may be sized and shaped to allow the proximal head portion 622 to be slid over the attachment arm 610 (as shown in FIG. 11B) while preventing the proximal head portion 622 from unintentionally disengaging from the attachment arm 610 prior to assembly with the soft tissue anchor 620.

The distal fixation end 624 may be generally “V” shaped or pointed, and may include a first barb 628a and a second barb 628b (collectively, 628). The distal fixation end 624 may be shaped such that proximal retraction of the soft tissue anchor 620 is prevented or hindered once the distal fixation end 624 is assembled with the frame 600 and/or body tissue. For example, the barbs 628 may each include a proximal end 630a, 630b (collectively, 630) radially spaced from the intermediate connecting portion or shank 626 and a distal end 632a, 632b (collectively, 632) in contact with or coupled to the distal fixation end 624. In some cases, fewer than two or more than two barbs 628 may be provided. In some cases, the proximal ends 630 of the barbs 628 may be configured to deflect radially inwards to facilitate distal advancement of the soft tissue anchor 620 through the frame 600, implant 12, and/or body tissue and resume an expanded outwardly extending configuration (as shown in FIG. 11A) after insertion, although this is not required. The distal fixation end 624 of the soft tissue anchor 620 is configured to lead the soft tissue anchor 620 as it is advanced into the bodily tissue. The barbs 628 are angled or sloped such that the soft tissue anchor 620 is easily distally advanced through the tissue but proximal retraction of the soft tissue anchor 620 is difficult or precluded. This may help ensure that once the soft tissue anchor 620 is assembled with the frame 600 and/or implant 12 and/or is implanted into the tissue it is self-anchoring. The angle and/or length of the barbs 628 may be varied It is contemplated that the barbs 628 may have structures or shapes different from an angled surface, including, but not limited to, curves, steps, etc. It is contemplated that other structural features in place of or in addition to the barbs 628 may be used to help secure the soft tissue anchor 620 to the frame 600 and implant 12 and/or within the tissue.

The intermediate connecting portion or shank 626 may be an elongate rod having a generally cylindrical shape; however, the intermediate connecting portion or shank 626 may take other shapes as desired. While the intermediate connecting portion or shank 626 is illustrated as having a generally uniform diameter cross-sectional dimension, it is contemplated that the cross-sectional dimension of the intermediate connecting portion or shank 626 may vary along a length thereof. For example, the intermediate connecting portion or shank 626 may decrease in cross-sectional dimension from the proximal head portion 622 towards the distal fixation end 624. This is just one example. Other configurations may be used, as desired. It is contemplated that the soft tissue anchor 620 may be formed as a single monolithic structure or a series of components affixed to one another, as desired.

It is further contemplated that the soft tissue anchor 620 may take other configurations. For example, the soft tissue anchor 620 may be a barbed “U” shaped tissue anchor similar to a staple, having two distally extending legs. In such an instance, the barbed edges or legs penetrate and secure within the tissue while the connecting portion or bridge of the “U” provides a channel to engage the attachment arm 610 and provides a buttress to hold down the implant 12 between the barbed legs. In other embodiments, the soft tissue anchor 620 may have a threaded distal end region. The threaded region may penetrate the tissue while the head of the screw provides a buttress to hold down the implant. This is just an example.

The attachment arm 610 may include a raised detent 612 positioned adjacent a free end 614 thereof. The detent 612 may extend from a first surface 606 (e.g., surface facing away from the implant 12) of the frame 600 in a direction away from the implant 12. However, it is contemplated that the detent 612 may extend from a second surface 608 (e.g., surface facing towards the implant 12) of the frame 600 in a direction towards the implant 12. In some embodiments, detents 612 may be provided on both the first and second surfaces 606, 608. The detent 612 may include a distal side 616 and a proximal side 618. In some cases, the distal side 616 may include a ramped surface that increases in height in the proximal direction. For example, the distal side 616 may increase in height in a curved or sloped manner. This may facilitate assembly of the soft tissue anchor 620 with the attachment arm 610. In some cases, the proximal side 618 may extend at an angle orthogonal to the first surface 606 of the attachment arm 610. In other cases, the proximal side 618 may have a curve or slope which is steeper than the distal side 616. The proximal side 618 may be configured to provide a mechanical stop between the first end 644 of the proximal head portion of the soft tissue anchor 620 and the detent 612. It is contemplated that the detent 612 may be configured to deform or flex when a force is applied to the attachment arm 610 to allow the soft tissue anchor 620 to decouple from the attachment arm 610.

To assemble the soft tissue anchor 620 with the attachment arm 610, the free end 614 of the attachment arm 610 may be inserted into the channel 642 of the proximal head portion 622 of the soft tissue anchor 620 adjacent the second end 646 thereof. The attachment arm 610 may be advanced within the channel 642 until the detent 612 is positioned outside of the channel 642 adjacent to the first end 644 of the proximal head portion 622. Alternatively, the soft tissue anchor 620 may be advanced relative to the attachment arm 610 until the detent 612 is positioned outside of the channel 642 adjacent to the first side of the proximal head portion 622. The channel 642 of the proximal head portion 622 of the soft tissue anchor 620 may have a width 648 that is greater than the width 650 of the attachment arm 610. The width 648 of the channel 642 may be greater than the width 650 of the attachment arm 610 so that the soft tissue anchor 620 may freely slide over that portion of the attachment arm 610. Alternatively, the width 648 of the channel 642 may be approximately the same as the width 650 of the attachment arm 610 to provide a friction fit and further secure the soft tissue anchor 620 relative to the attachment arm 610. The implant 12 may be affixed adjacent to the second surface 608 of the frame 600 simultaneously with or subsequent to the assembly of the soft tissue anchors 620 therewith. It is contemplated that the soft tissue anchors 620 may penetrate or pierce the implant 12 (see, for example, FIG. 11B) to secure the implant 12 to the frame 600 for delivery to a target site.

The frame 600, implant 12, and soft tissue anchor 620 may be delivered to the body using a delivery system and method similar to that described above with respect to FIGS. 2-4. Once the implant 12 is positioned at the target location, it may be temporarily secured, using, for example, a bone tack. The delivery system may be released and retracted from the portal to allow other devices to be inserted. In some cases, a tether may be coupled to the frame 600 and extend out of the body to facilitate retrieval of the frame 600 once it is uncoupled from the implant 12. A tool may then be advanced to decouple the soft tissue anchor 620 from the frame 600. In some cases, the tool may be similar to a punch type tool, although this is not required. The tool may be aligned with the proximal head portion 622 of the soft tissue anchor 620. A force may then be applied to distally advance the soft tissue anchor 620 such that the distal fixation end 624 or distal tip pierces the tissue. The tool may be repositioned to align with another soft tissue anchor 620 and a force exerted on the soft tissue anchor 620 to decouple it from the frame 600. This may be repeated for each soft tissue anchor 620. This may allow multiple soft tissue anchors 620 to be deployed without requiring the clinician to individually load each soft tissue anchor 620 into the tool and to repeatedly insert and remove the tool. Once each soft tissue anchor 620 has been driven into the tissue, the frame 600 may be retracted from the body. The retraction of the frame 600 may exert a pulling force on the attachment arms 610 which in turn may cause the detent 612 to deform against the first end 644 of the proximal head portion 622 thus allowing the attachment arms 610 to pass through the channel 642 of the proximal head portion 622 of the soft tissue anchors 620 and releasing the frame 600 from the soft tissue anchors 620.

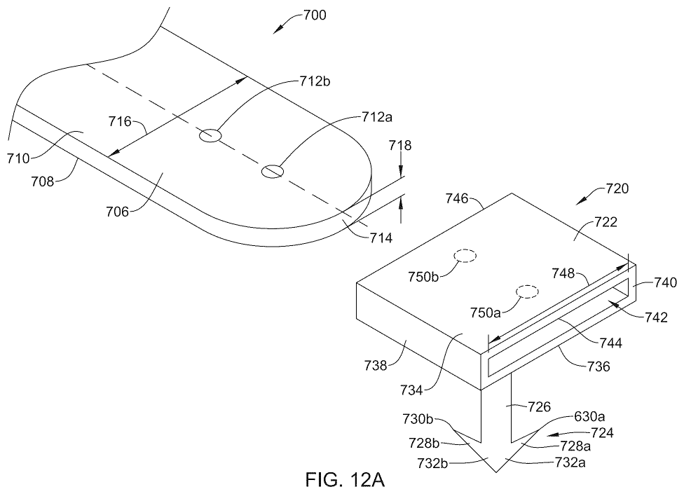

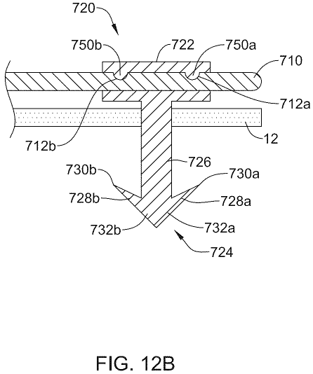

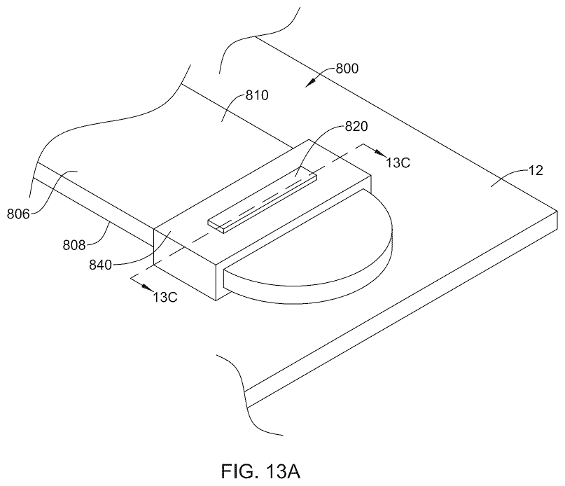

FIG. 12A illustrates an exploded perspective view of another illustrative attachment arm 710 of a frame 700 and soft tissue anchor 720. FIG. 12B is a partial cross-sectional view of the illustrative attachment arm 710 of a frame 700 assembled with the soft tissue anchor 720 and an implant 12. The frame 700 may be similar in form and function to the frame 100 described herein. The soft tissue anchor 720 may be configured to be assembled with the frame 700 and an implant 12 (not explicitly shown) to releasably secure the implant 12 to the frame 700. While the soft tissue anchor 720 is described with respect to a single attachment arm 710, it should be understood a soft tissue anchor 720 may be provided in any number of attachment arms 710, as desired.

The soft tissue anchor 720 may include a proximal head portion 722 configured to hold down the implant 12 and a distal fixation end 724 configured to penetrate the tissue. An elongate intermediate connecting portion or shank 726 may extend between the proximal head portion 722 and the distal fixation end 724. It is contemplated that the proximal head portion 722 may have a width greater than a width of the intermediate connecting portion 726. The proximal head portion 722 may include a top side 734 that faces away from the implant 12 when the implant 12 is attached to the frame 700, a bottom side 736 that faces or is juxtaposed with the implant 12 when the implant 12 is attached to the frame 700, a first lateral side 738 extending between the top side 734 and the bottom side 736, and a second lateral side 740 opposite the first lateral side 738 and extending between the top side 734 and the bottom side 736. Collectively, the top side 734, bottom side 736, first lateral side 738, and second lateral side 740 may define a channel 742 that extends from a first end 744 to a second end 746 of the proximal head portion 722. The channel 742 may be sized and shaped to allow the proximal head portion 722 to be slid over the attachment arm 710 (as shown in FIG. 12B) while preventing the proximal head portion 722 from unintentionally disengaging from the attachment arm 710 prior to assembly with the soft tissue anchor 720. A first detent 750a and a second detent 750b (collectively, 750) may extend into the channel 742 from the top side 734. Detents 750 may be considered protuberances. Alternatively, or additionally, detents 750 may extend into the channel 742 from the bottom side 736 (not explicitly shown) and/or the lateral sides 738, 740. It is contemplated that fewer than two (e.g., one) or greater than two (e.g., three, four, five, or more) detents 750 may extend from a surface of the proximal head portion 722 into the channel 742. In other instances, detents 750 may extend from a tab or other portion of the proximal head portion 722 without extending into the channel 742. While the detents 750 are illustrated as having a generally hemispherical shape, it is contemplated that the detents 750 may take any shape desired, including, but not limited to, conical, cubical, pyramidal, etc. Further, while the detents 750 are illustrated as being positioned in a linear arrangement, this is not required. The detents 750 may be positioned in any configuration, symmetrical or asymmetrical, desired.

The distal fixation end 724 may be generally “V” shaped or pointed, and may include a first barb 728a and a second barb 728b (collectively, 728). The distal fixation end 724 may be shaped such that proximal retraction of the soft tissue anchor 720 is prevented or hindered once the distal fixation end 724 is assembled with the frame 700 and/or body tissue. For example, the barbs 728 may each include a proximal end 730a, 730b (collectively, 730) radially spaced from the intermediate connecting portion or shank 726 and a distal end 732a, 732b (collectively, 732) in contact with or coupled to the distal fixation end 724. In some cases, fewer than two or more than two barbs 728 may be provided. In some cases, the proximal ends 730 of the barbs 728 may be configured to deflect radially inwards to facilitate distal advancement of the soft tissue anchor 720 through the frame 700, implant 12, and/or body tissue and resume an expanded outwardly extending configuration (as shown in FIG. 12A) after insertion, although this is not required. The distal fixation end 724 or distal tip of the soft tissue anchor 720 is configured to lead the soft tissue anchor 720 as it is advanced into the bodily tissue. The barbs 728 are angled or sloped such that the soft tissue anchor 720 is easily distally advanced through the tissue but proximal retraction of the soft tissue anchor 720 is difficult or precluded. This may help ensure that once the soft tissue anchor 720 is assembled with the frame 700 and/or implant 12 and/or is implanted into the tissue it is self-anchoring. The angle and/or length of the barbs 728 may be varied It is contemplated that the barbs 728 may have structures or shapes different from an angled surface, including, but not limited to, curves, steps, etc. It is contemplated that other structural features in place of or in addition to the barbs 728 may be used to help secure the soft tissue anchor 720 to the frame 700 and implant 12 and/or within the tissue.

The intermediate connecting portion or shank 726 may be an elongate rod having a generally cylindrical shape; however, the intermediate connecting portion or shank 726 may take other shapes as desired. While the intermediate connecting portion or shank 726 is illustrated as having a generally uniform diameter cross-sectional dimension, it is contemplated that the cross-sectional dimension of the intermediate connecting portion or shank 726 may vary along a length thereof. For example, the intermediate connecting portion or shank 726 may decrease in cross-sectional dimension from the proximal head portion 722 towards the distal fixation end 724. This is just one example. Other configurations may be used, as desired. It is contemplated that the soft tissue anchor 720 may be formed as a single monolithic structure or a series of components affixed to one another, as desired.

It is further contemplated that the soft tissue anchor 720 may take other configurations. For example, the soft tissue anchor 720 may be a barbed “U” shaped tissue anchor similar to a staple, having two distally extending legs. In such an instance, the barbed edges or legs penetrate and secure within the tissue while the connecting portion or bridge of the “U” provides a channel to engage the attachment arm 710 and provides a buttress to hold down the implant 12 between the barbed legs. In other embodiments, the soft tissue anchor 720 may have a threaded distal end region. The threaded region may penetrate the tissue while the head of the screw provides a buttress to hold down the implant. This is just an example.

The attachment arm 710 includes a first surface 706 (e.g., surface facing away from the implant 12) and a second surface 708 (e.g., surface facing towards the implant 12) defining a thickness 718 therebetween. The attachment arm 710 may include a first hole or recess 712a positioned adjacent a free end 714 thereof and a second hole or recess 712b (collectively, 712) laterally spaced from the first recess 712a. In some cases, the recesses 712 may extend partially through the thickness 718 of the attachment arm 710. In other cases, the recesses 712 may extend entirely through the thickness 718 of the attachment arm 710 (e.g., from the first surface 706 to the second surface 708). It is further contemplated that some recesses 712 may extend partially through the thickness 718 while others extend entirely through the thickness 718 of the attachment arm 710. The attachment arm 710 may include fewer than two (e.g., one) or greater than two (e.g., three, four, five, or more) recesses 712. While the recesses 712 are illustrated as having a generally hemispherical shape to generally mate with the detents 750, it is contemplated that the recesses 712 may take any shape desired, including, but not limited to, conical, cubical, pyramidal, etc. Further, while the recesses 712 are illustrated as being positioned in a linear arrangement, this is not required. The recesses 712 may be positioned in any configuration, symmetrical or asymmetrical, desired. It is contemplated that the configuration of the recesses 712 may mirror or match the configuration of the detents 750 in the proximal head portion 722 of the soft tissue anchor 720.

To assemble the soft tissue anchor 720 with the attachment arm 710, the free end 714 of the attachment arm 710 may be inserted into the channel 742 of the proximal head portion 722 of the soft tissue anchor 720 adjacent the second end 746 thereof. The attachment arm 710 may be advanced within the channel 742 until the detents 750 of the proximal head portion 722 engage the recesses 712 of the attachment arm 710. For example, the first detent 750a of the soft tissue anchor 720 may be configured to be disposed within or otherwise engage with the first recess 712a of the attachment arm 710 and the second detent 750b of the soft tissue anchor 720 may be configured to be disposed within or otherwise engage with the second recess 712b of the attachment arm 710 to form a mechanical interlock between the two components, as shown in FIG. 12B. The channel 742 of the proximal head portion 722 of the soft tissue anchor 720 may have a width 748 that is greater than the width 716 of the attachment arm 710. The width 748 of the channel 742 may be greater than the width 716 of the attachment arm 710 so that the soft tissue anchor 720 may freely slide over that portion of the attachment arm 710. Alternatively, the width 748 of the channel 742 may be approximately the same as the width 716 of the attachment arm 710 to provide a friction fit and further secure the soft tissue anchor 720 relative to the attachment arm 710. The implant 12 may be affixed adjacent to the second surface 708 of the frame 700 simultaneously with or subsequent to the assembly of the soft tissue anchors 720 therewith. It is contemplated that the soft tissue anchors 720 may penetrate or pierce the implant 12 (see, for example, FIG. 12B) to secure the implant 12 to the frame 700.

It is contemplated that positioning the detents 750 and recesses 712 at staggered positions about the width 748 of the of the channel 742 and the width 716 of the attachment arm 710 may help facilitate assembly by preventing a detent (e.g., detent 750b) from prematurely engaging with a recess (e.g., recess 712a) intended to correspond to a different detent (e.g., detent 750a), although this is not required. For example, a first recess 712a may be laterally offset from the longitudinal axis 702 of the attachment arm 710 in a first direction and the second recess 712b may be laterally offset from the longitudinal axis 702 of the attachment arm 710 in a second direction opposite the first direction. The corresponding detents 750 on the proximal head portion 722 may be similarly arranged such that the detents 750 may be assembled with the recesses 712.

The frame 700, implant 12, and soft tissue anchor 720 may be delivered to the body using a delivery system and method similar to that described above with respect to FIGS. 2-4. Once the implant 12 is positioned at the target location, it may be temporarily secured, using, for example, a bone tack. The delivery system may be released and retracted from the portal to allow other devices to be inserted. In some cases, a tether may be coupled to the frame 700 and extend out of the body to facilitate retrieval of the frame 700 once it is uncoupled from the implant 12. A tool may then be advanced to decouple the soft tissue anchor 720 from the frame 700. In some cases, the tool may be similar to a punch type tool, although this is not required. The tool may be aligned with the proximal head portion 722 of the soft tissue anchor 720. A force may then be applied to distally advance the soft tissue anchor 720 such that the distal fixation end 724 or distal tip pierces the tissue. The tool may be repositioned to align with another soft tissue anchor 720 and a force exerted on the soft tissue anchor 720 to decouple it from the frame 700. This may be repeated for each soft tissue anchor 720. This may allow multiple soft tissue anchors 720 to be deployed without requiring the clinician to individually load each soft tissue anchor 720 into the tool and to repeatedly insert and remove the tool. Once each soft tissue anchor 720 has been driven into the tissue, the frame 700 may be retracted from the body. The retraction of the frame 700 may exert a pulling force on the attachment arms 710 which in turn may cause the detents 750 to disengage from the recesses 712 thus allowing the attachment arms 710 to pass through the channel 742 of the proximal head portion 722 of the soft tissue anchors 720 and releasing the frame 700 from the soft tissue anchors 720.

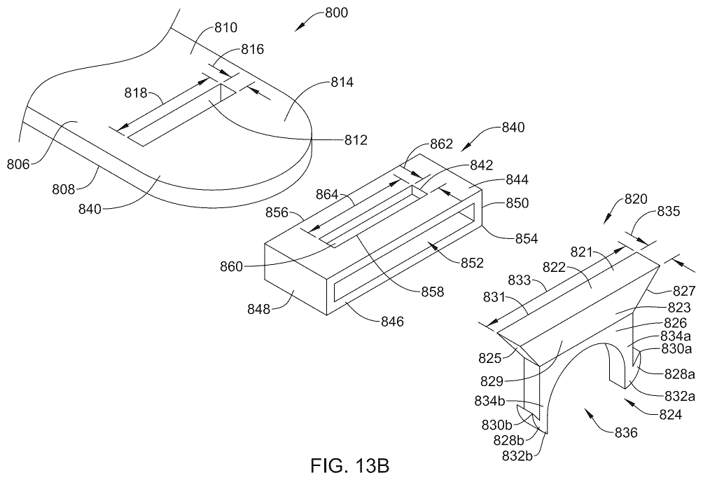

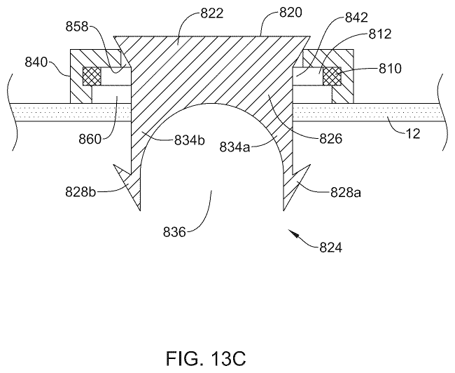

FIG. 13A illustrates a perspective view of another illustrative attachment arm 810 of a frame 800 and soft tissue anchor 820, with an implant 12 attached thereto. FIG. 13B is an exploded perspective view of the illustrative attachment arm 810 of a frame 800, retainer 840 and soft tissue anchor 820 of FIG. 13A. The frame 800 may be similar in form and function to the frame 100 described herein. The soft tissue anchor 820 may be configured to be assembled with the frame 800 and an implant 12 to releasably secure the implant to the frame 800. FIG. 13C is a cross-sectional view of the illustrative attachment arm 810, taken at line 13C-13C of FIG. 13A. While the soft tissue anchor 820 is described with respect to a single attachment arm 810, it should be understood a soft tissue anchor 820 may be provided in any number of attachment arms 810, as desired.

In the illustrative embodiment of FIGS. 13A-13C, the soft tissue anchor 820 may be removably coupled to the attachment arm 810 via a retainer 840. The retainer 840 may be configured to be slid over the free end 814 of the attachment arm 810. Generally, the soft tissue anchor 820 may be advanced through an aperture 842 in the retainer 840 and an aperture 812 in the attachment arm 810. The retainer 840 may include features configured to engage a mating feature of the soft tissue anchor 820 to releasably secure the soft tissue anchor 820 thereto, although this is not required.

The soft tissue anchor 820 may include a proximal head portion 822 configured to hold down the implant 12 and a distal fixation end 824 configured to penetrate the tissue. An elongate intermediate connecting portion 826 may extend between the proximal head portion 822 and the distal fixation end 824. In some instances, the proximal head portion 822 may include one or more, or a plurality of beveled surfaces. The proximal head portion 822 may take the form of a truncated pyramid. The proximal head portion 822 may have an upper face 821 and a lower face 823. The upper face 821 may have a greater length 833 and width 835 than the lower face 823, although this is not required. The lower face 823 may be connected to or positioned adjacent to the intermediate connection portion 826. The proximal head portion 822 may further include a first lateral side face 825, a second lateral side face 827 opposite the first lateral side face 825, a third lateral side face 829, and a fourth lateral side face 831 opposite the third lateral side face 829. In some instances, the lateral side faces may be beveled at an acute angle to the insertion direction of the soft tissue anchor 820. The proximal head portion 822 may take other shapes as desired, such as, but not limited, rectangular prism, cuboid, ellipsoid, etc.

The distal fixation end 824 may have barbed “U” shape including a first leg 834a and a second leg 834b (collectively, 834) extending from the intermediate connection portion 826. The legs 834 may be laterally spaced to define a channel 836 therebetween. The first leg 834a may include a distal tip including a first barb 828a and the second leg 834b may include a distal tip including a second barb 828b (collectively, 828). The distal fixation end 824 may be shaped such that proximal retraction of the soft tissue anchor 820 is prevented or hindered once the distal fixation end 824 is assembled with the frame 800 and/or body tissue. For example, the barbs 828 may each include a proximal end 830a, 830b (collectively, 830) radially spaced from the respective leg 834 and a distal end 832a, 832b (collectively, 832) in contact with or coupled to the distal fixation end 824. In some cases, fewer than two or more than two barbs 828 may be provided. In some cases, the proximal ends 830 of the barbs 828 may be configured to deflect radially inwards to facilitate distal advancement of the soft tissue anchor 820 through the frame 800, retainer 840, implant 12, and/or body tissue and resume an expanded outwardly extending configuration (as shown in FIGS. 13B and 13C) after insertion, although this is not required. The distal fixation end 824 or distal tips of the legs 834 of the soft tissue anchor 820 is configured to lead the soft tissue anchor 820 as it is advanced into the bodily tissue. The barbs 828 are angled or sloped such that the soft tissue anchor 820 is easily distally advanced through the tissue but proximal retraction of the soft tissue anchor 820 is difficult or precluded. This may help ensure that once the soft tissue anchor 820 is assembled with the frame 800 and/or implant 12 and/or is implanted into the tissue it is self-anchoring. The angle and/or length of the barbs 828 may be varied It is contemplated that the barbs 828 may have structures or shapes different from an angled surface, including, but not limited to, curves, steps, etc. It is contemplated that other structural features in place of or in addition to the barbs 828 may be used to help secure the soft tissue anchor 820 to the frame 800 and implant 12 and/or within the tissue.

The intermediate connecting portion 826 may couple the proximal head portion 822 and the distal fixation end 824. In some cases, the intermediate connection portion 826 may form a bridge between the first and second legs 834a, 834b. It is contemplated that the soft tissue anchor 820 may be formed as a single monolithic structure or a series of components affixed to one another, as desired.

It is further contemplated that the soft tissue anchor 820 may take other configurations. For example, the soft tissue anchor 820 may be any of the soft tissue anchors described herein. In other embodiments, the soft tissue anchor 820 may be a screw having a threaded distal end region. The threaded region may penetrate the tissue while the head of the screw provides a buttress to hold down the implant. This is just an example.

The attachment arm 810 may include an aperture 812 configured to receive a portion of the soft tissue anchor 820. The aperture 812 may have a rectangular cross-sectional shape similar to the general cross-sectional shape of the proximal head portion 822 of the soft tissue anchor 820 and extend through a thickness of the attachment arm 810 from a first surface 806 that faces away from the implant 12 when the implant 12 is attached to the frame 800 (e.g., a first surface that faces away from a target site in the body) and a second surface 808 that faces the example implant 12 (e.g., a second surface that faces a target site in the body). The aperture 812 may be sized and shaped to allow the proximal head portion 822 of the soft tissue anchor 820 to pass therethrough. For example, the aperture 812 may have a width 816 that is greater than the width 835 of the proximal head portion 822 and a length 818 that is greater than the length 833 of the proximal head portion 822.

The retainer 840 may include a top side 844 that faces away from the implant 12 when the implant 12 is attached to the frame 800, a bottom side 846 that faces the implant 12 when the implant 12 is attached to the frame 800, a first lateral side 848 extending between the top side 844 and the bottom side 846, and a second lateral side 850 opposite the first lateral side and extending between the top side 844 and the bottom side 846. Collectively, the top side 844, bottom side 846, first lateral side 848, and second lateral side 850 may define a channel 852 that extends from a first end 854 to a second end 856 of the retainer 840. The channel 852 may be sized and shaped to allow the retainer 840 to be slid over the attachment arm 810 (as shown in FIG. 13A) while preventing the retainer 840 from unintentionally disengaging from the attachment arm 810 prior to assembly with the soft tissue anchor 820. The retainer 840 may further include a first opening 858 in the top side 844 and a second opening 860 defined in the bottom side 846. The first and second openings 585, 860 may have a rectangular cross-sectional shape similar to the general cross-sectional shape of the proximal head portion 822 of the soft tissue anchor 820, although this is not required. The first and second openings 858, 860 may be generally aligned to define an aperture 842 extending from the top side 844 to the bottom side 846. The first and second openings 858, 860 may have similar or the same diameters or may have differing diameters, as desired. In some embodiments, the first opening 858 may have a width 862 that is less than the width 835 of the proximal head portion 822 and a length 864 that is less than the length 833 of the proximal head portion 822 while the second opening 860 may have a width that is greater than the width 835 of the proximal head portion 822 and a length 864 that is greater than the length 833 of the proximal head portion 822

To assemble the soft tissue anchor 820 with the attachment arm 810, the retainer 840 may be advanced over the free end 814 of the attachment arm 810 until the aperture 842 of the retainer 840 is generally aligned with the aperture 812 of the attachment arm 810. The distal fixation end 824 of the soft tissue anchor 820 may then be advanced through the apertures 812, 842 in a direction from the top side 844 towards the bottom side 846 of the retainer 840. The soft tissue anchor 820 may be advanced until the first, second, third, and fourth faces 825, 827, 829, 831 engage the side walls of the first opening 858 in the retainer 840. The faces 825, 827, 829, 831 may form a friction fit with the first opening 858 such that a first force secures the soft tissue anchor 820 within the first opening 858 and a second force greater than the first forces pushes the soft tissue anchor 820 through the first opening 858, the aperture 842 and the second opening 860. It is contemplated that the implant 12 may be positioned adjacent to the second surface 808 of the frame 800 and/or the bottom side 846 of the retainer 840 during assembly such that the soft tissue anchor 820 pierces the implant 12 and secures it to the frame 800.

The frame 800, implant 12, and soft tissue anchor 820 may be delivered to the body using a delivery system and method similar to that described above with respect to FIGS. 2-4. Once the implant 12 is positioned at the target location, it may be temporarily secured, using, for example, a bone tack. The delivery system may be released and retracted from the portal to allow other devices to be inserted. In some cases, a tether may be coupled to the frame 800 and extend out of the body to facilitate retrieval of the frame 800 once it is uncoupled from the implant 12. A tool may then be advanced to decouple the soft tissue anchor 820 from the frame 800. In some cases, the tool may be similar to a punch type tool, although this is not required. The tool may be aligned with the proximal head portion 822 of the soft tissue anchor 820. A force may then be applied to distally advance the soft tissue anchor 820 such that the distal fixation end 824 or distal tips of the legs pierces the tissue. It is contemplated that the top side 844 of the retainer 840 may flex, deflect, deform, or fracture as the force is applied to the proximal head portion 822 allowing the proximal head portion 822 to pass through or disengage from the first opening 858. In some cases, the angle or sloped surface of the first, second, third, and fourth faces 825, 827, 829, 831 may facilitate passage of the proximal head portion 822 through the first opening 858. As noted above, the second opening 860 may be sized such that the proximal head portion 822 easily passes therethrough, although this is not required. The tool may be repositioned to align with another soft tissue anchor 820 and a force exerted on the soft tissue anchor 820 to decouple it from the frame 800. This may be repeated for each soft tissue anchor 820. This may allow multiple soft tissue anchors 820 to be deployed without requiring the clinician to individually load each soft tissue anchor 820 into the tool and to repeatedly insert and remove the tool.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An implant delivery system, the implant delivery system comprising:
a delivery shaft including a proximal portion and a distal portion; and
a frame coupled to the distal portion of the delivery shaft, wherein the frame includes a body portion and a plurality of attachment arms extending away from the body portion; and
at least one soft tissue anchor releasably secured to a free end of at least one of the plurality of attachment arms; and
an implant releasably secured to the frame via the at least one soft tissue anchor.

2. The implant delivery system of claim 1, wherein the at least one soft tissue anchor is configured to remain in a body with the implant.

3. The implant delivery system of claim 1, wherein the at least one tissue anchor comprises a proximal head portion, a distal fixation end, and an intermediate connecting portion extending between the proximal head portion and the distal fixation end.

4. The implant delivery system of claim 3, wherein the proximal head portion is generally disc-shaped.

5. The implant delivery system of claim 3, wherein the distal fixation end comprises a first barb and a second barb.

6. The implant delivery system of claim 3, wherein the intermediate connection portion comprises an elongate rod.

7. The implant delivery system of claim 3, further comprising a recess formed in the at least one of the plurality of attachment arms adjacent to the free end thereof.

8. The implant delivery system of claim 7, wherein the recess extends partially through a thickness of the at least one of the plurality of attachment arms to define a reduced thickness region.

9. The implant delivery system of claim 8, further comprising a through hole extending through the reduced thickness region.

10. The implant delivery system of claim 7, wherein the recess is configured to receive the proximal head portion of the at least one soft tissue anchor therein.

11. The implant delivery system of claim 10, wherein the intermediate connection portion extends through the through hole.

12. The implant delivery system of claim 10, wherein under an applied force, the proximal head portion of the at least one soft tissue anchor is configured to push through the reduced thickness region of the at least one of the plurality of attachment arms to release the at least one soft tissue anchor from the frame.

13. The implant delivery system of claim 12, wherein the reduced thickness region is configured to fracture.

14. The implant delivery system of claim 12, wherein the reduced thickness region is configured to deform to allow the proximal head portion to pass through the through hole.

15. The implant delivery system of claim 3, wherein the proximal head portion further comprises one or more radially extending tabs.

16. The implant delivery system of claim 15, further comprising an aperture formed through the at least one of the plurality of attachment arms adjacent the free end thereof.

17. The implant delivery system of claim 16, where the aperture extends through a thickness of the at least one of the plurality of attachment arms.

18. The implant delivery system of claim 16, further comprising a circumferentially extending groove formed in a side wall of aperture, the circumferentially extending groove positioned between a first surface of the at least one of the plurality of attachment arms and a second surface of the at least one of the plurality of attachment arms.

19. The implant delivery system of claim 18, further comprising at least one axially extending slot formed in a side wall of aperture, the at least one axially extending slot extending from the second surface of the at least one of the plurality of attachment arms to the circumferentially extending groove.

20. The implant delivery system of claim 19, wherein during assembly of the at least one soft tissue anchor with the at least one of the plurality of attachment arms and deployment of the at least one soft tissue anchor from the at least one of the plurality of attachment arms, the one or more radially extending tabs are configured to slide within the at least one axially extending slot.

21. The implant delivery system of claim 18, wherein the one or more radially extending tabs are rotatably disposed within the circumferentially extending groove during delivery of the implant to a target location.

22. The implant delivery system of claim 3, wherein the proximal head portion further comprises one or more radially extending detents.

23. The implant delivery system of claim 22, wherein the detent extends about a circumference of the proximal head portion.

24. The implant delivery system of claim 22, further comprising an aperture formed through the at least one of the plurality of attachment arms adjacent the free end thereof.

25. The implant delivery system of claim 24, where the aperture extends through a thickness of the at least one of the plurality of attachment arms.

26. The implant delivery system of claim 24, further comprising a retainer, the retainer configured to slide over a free end of the at least one of the plurality of attachment arms.

27. The implant delivery system of claim 26, wherein the retainer comprises a top side, a bottom side, a first lateral side extending between the top side and the bottom side, and a second lateral side opposite the first lateral side and extending between the top side and the bottom side.

28. The implant delivery system of claim 27, wherein collectively the top side, the bottom side, the first lateral side, and the second lateral side define a channel that extends from a first end to a second end of the retainer, the channel configured to receive the at least one of the plurality of attachment arms.

29. The implant delivery system of claim 27, further comprising a first opening in the top side of the retainer and a second opening defined in the bottom side of the retainer, wherein the first opening has a first diameter and the second opening has a second diameter, the second diameter greater than the first diameter.

30. The implant delivery system of claim 29, wherein the first opening and the second opening of the retainer are aligned with the aperture of the at least one of the plurality of attachment arms and the one or more radially extending detents of the proximal head portion are received within a mating recess formed in a side wall of the first opening of the retainer.
